(19) United States
(12) Patent Application Publication
(10) Pub. No.: US 2011/0012096 A1
(43) Pub. Date: Jan. 20, 2011

(54) PHOTOACTIVE NANOSTRUCTURE AND METHOD OF MANUFACTURING SAME

(75) Inventors: Chanoch Carmeli, Tel-Aviv (IL); Itai Carmeli, Tel-Aviv (IL); Ludmila Frolov, Rishon-LeZion (IL); Shachar Richter, Mazkeret Batia (IL); Yossi Rosenwaks, Hod-HaSharon (IL); Alexander Govorov, Athens, OH (US)

Correspondence Address: MARTIN D. MOYNIHAN d/b/a PRTSI, INC., P.O. BOX 16446, ARLINGTON, VA, 22215, US

(73) Assignees: RAMOT AT TEL-AVIV UNIVERSITY LTD., TEL-AVIV (IL); OHIO UNIVERSITY OFFICES OF INNOVATION CENTER AND T, ATHENS, OH (US)

(21) Appl. No.: 12/918,352

(22) PCT Filed: Feb. 19, 2009
(86) PCT No.: PCT/IL09/00196

§ 371 Date: Aug. 19, 2010

Related U.S. Application Data
(60) Provisional application No. 61/064,168, filed on Feb. 20, 2008.

(51) Int. Cl. H01L 51/46; C07K 14/415; C07K 14/195; H01L 51/48

(52) U.S. Cl. 257/40; 530/402; 438/1; 977/773; 977/936; 257/E51.045

(57) ABSTRACT

A nanostructure comprising at least one semiconductor nanoparticle bound to a photocatalytic unit of a photosynthetic organism is disclosed. The nanoparticle and a binding between the nanoparticle and the photocatalytic unit are selected such that transfer of electrons from the photocatalytic unit to the nanoparticle is prevented or suppressed relative to transfer of excitons from the nanoparticle to the photocatalytic unit. Uses of same and methods of fabricating devices with same are also disclosed. Nanostructures comprising electrically conductive nanoparticles are also disclosed.

### RELATED APPLICATION/S

This application claims the benefit of priority from U.S. Patent Application No. 61/064,168 filed Feb. 20, 2008, the contents of which are incorporated by reference as if fully set forth herein.

### FIELD AND BACKGROUND OF THE INVENTION

The present invention, in some embodiments thereof, relates to a photoactive nanostructure comprising one or more solid nanoparticles bound to a photocatalytic unit. The present invention also relates to fabrication of devices with multi-layers of photocatalytic units.

Nanoscience is the science of small particles of materials and is one of the most important research frontiers in modern technology. These small particles are of interest from a fundamental point of view since they enable construction of materials and structures of well-defined properties. With the ability to precisely control material properties arise new opportunities for technological and commercial development and applications of nanoparticles have been shown or proposed in areas as diverse as micro- and nanoelectronics, nanofluidics, coatings and paints and biotechnology.

It is well established that future development of microelectronics, magnetic recording devices and chemical sensors will be achieved by increasing the packing density of device components. Traditionally, microscopic devices have been formed from larger objects, but as these products get smaller, below the micron level, this process becomes increasingly difficult. It is therefore appreciated that the opposite approach is to be employed, essentially, the building of microscopic devices from a molecular level up, primarily via objects of nanometric dimensions.

Fabrication of molecular circuits is presently beyond the resolution of conventional patterning techniques such as electron beam lithography. However, positioning of molecules with sub nanometer precision is routine in nature, and crucial to the operation of biological complexes such as photosynthetic complexes.

Green plants, cyanobacteria and photosynthetic bacteria capture and utilize sunlight by means of molecular electronic complexes, reaction centers that are embedded in their membranes. In oxygenic plants and cyanobacteria, photon capture and conversion of light energy into chemical energy take place in specialized membranes called thylakoids. The thylakoids are located in chloroplast in higher plants or consists of foldings of the cytoplasmic membrane in cyanobacteria. The thylakoids, consisting of stacked membrane disks (called grana) and unstacked membrane disks (called stroma). The thylakoid membrane contains two key photosynthetic components, photosystem I and photosystem II, designated PS I and PS II, respectively. Photosynthesis requires PSII and PSI working in sequence, using water as the source of electrons and CO₂ as the terminal electron acceptor.

PS I is a transmembrane multisubunit protein-chlorophyll complex that mediates vectorial light-induced electron transfer. The nano-size dimension, an energy yield of approximately 58% and the quantum efficiency of almost 1 [K. Brettel, Biochim. Biophys. Acta 1997, 1318 322-373] makes the reaction center a promising unit for applications in molecular nano-electronics. PS I mediates light-induced electron transfer from plastocyanin or cytochrome C₅₅₃ to ferredoxin.

Since the PS I reaction center is a pigment-protein complex responsible for the photosynthetic conversion of light energy to chemical energy, these reaction centers may be used as electronic components in a variety of different devices. These possible devices include, but are not limited to, spatial imaging devices, solar batteries, optical computing and logic gates, optoelectronic switches, photonic A/D converters, and thin film “flexible” photovoltaic structures. However, in order to incorporate these PS I reaction centers into molecular devices, it is essential to immobilize the PSI reaction centers onto a substrate without their denaturation.

International Patent Publication No. WO2006/090381, the contents of which are hereby incorporated by reference discloses a technique for mutating a polypeptide of a photocatalytic unit, such that the amino acid sequence of the polypeptide mediates covalent attachment of the photocatalytic unit to a solid surface. The photocatalytic activity of the photocatalytic unit is maintained after the attachment.

International Patent Publication No. WO2008/023373 discloses a technique in which a modified photocatalytic unit is attached to a semiconductor solid material, in a manner such that when light is absorbed by the photocatalytic unit, an electric field is generated at sufficient amount to induce charge carrier locomotion within the semiconductor surface.

International Patent Publication No. WO2008/023372 discloses a technique in which photocatalytic units of a photosynthetic organism are covalently attached to an electrode to provide a layer of photoactive nanoparticles on the electrode. Another electrode is deposited on the layer of photoactive nanoparticles.

### SUMMARY OF THE INVENTION

According to an aspect of some embodiments of the present invention there is provided a nanostructure. The nanostructure comprises at least one semiconductor nanoparticle bound to a photocatalytic unit of a photosynthetic organism. In various exemplary embodiments of the invention the nanoparticle and the binding between the nanoparticle and the photocatalytic unit are selected such that transfer of electrons from the photocatalytic unit to the nanoparticle is prevented or suppressed relative to transfer of excitons from the nanoparticle to the photocatalytic unit.

According to an aspect of some embodiments of the present invention there is provided a device which comprises the nanostructure described herein attached to at least one electrode.

According to some embodiments of the invention, the at least one semiconductor nanoparticle binds to a polypeptide of a reaction center of the photocatalytic unit.

According to some embodiments of the invention, the at least one semiconductor nanoparticle binds to an antenna chlorophyll of the photocatalytic unit.

According to some embodiments of the invention, the photosynthetic organism is a green plant.

According to some embodiments of the invention, the photosynthetic organism is a cyanobacteria.

According to some embodiments of the invention, the photocatalytic unit is photosystem I (PS I).

According to some embodiments of the invention, the photosynthetic organism is a Synechosystis sp. PCC 6803.

According to some embodiments of the invention, the at least one semiconductor nanoparticle is bound to an electron acceptor side of the reaction center.

According to some embodiments of the invention, the at least one semiconductor nanoparticle binds to an electron donor side of the reaction center.

According to some embodiments of the invention, the at least one semiconductor nanoparticle is binds to the photocatalytic unit via a bifunctional connecting molecule.

According to some embodiments of the invention, the bifunctional connecting molecule is attached to a free carboxyl of the photocatalytic unit.

According to some embodiments of the invention, the bifunctional connecting molecule is attached to a free primary amine of the photocatalytic unit.

According to some embodiments of the invention, the bifunctional connecting molecule comprises a succinylimide moiety

According to some embodiments of the invention, a polypeptide of the photocatalytic unit comprises at least one substitution mutation.

According to some embodiments of the invention, the substitution mutation is on an extra-membrane loop of the photocatalytic unit.

According to some embodiments of the invention, the polypeptide is Photosystem I P700 chlorophyll a apoprotein A2 (psa B).

According to some embodiments of the invention, the Psa B comprises a substitution mutation in at least one position demarked by the coordinates D236C, S247C, D480C, S500C, S600C, Y635C.

According to some embodiments of the invention, the at least one substitution mutation is cysteine.

According to some embodiments of the invention, the polypeptide comprises an amino acid sequence is as set forth in SEQ ID NOs: 1, 2, 3, 4, 5 and 6.

According to some embodiments of the invention, a diameter of the at least one semiconductor nanoparticle is about 2 nm to 20 nm.

According to some embodiments of the invention, a diameter of the at least one semiconductor nanoparticle is about 8 nm.

According to some embodiments of the invention, a length of the bifunctional connecting molecule is about 1.5 nm.

According to some embodiments of the invention, the semiconductor nanoparticle is selected from the group consisting of a CdTe nanoparticle, a CdSe nanoparticle, and a CdS nanoparticle.

According to some embodiments of the invention, the electrode comprises a transition metal.

According to some embodiments of the invention, the transition metal is selected from the group consisting of silver, gold, copper, platinum, nickel, aluminum and palladium.

According to some embodiments of the invention, the device serves as a component selected from the group consisting of a photodiode, a phototransistor, a logic gate, a solar cell and an optocoupler.

According to an aspect of some embodiments of the present invention there is provided a method of fabricating a device, the method comprising: (a) covalently attaching photosystem I (PSI) of a photosynthetic organism to a solid support to generate a monolayer of the photocatalytic units; (b) depositing platinum ions on the monolayer under conditions that allow generation of a platinized monolayer of the photocatalytic units: (c) depositing free, pre-plantinized PSIs of the photosynthetic organism on the monolayer of the photocatalytic units to generate a multilayered assembly of the photocatalytic units, wherein a polypeptide of the pre-platinized PSIs comprise at least one cysteine substitution mutation, thereby fabricating the device.

According to some embodiments of the invention, the conditions comprise incubation in light in the presence of an electron donor.

According to some embodiments of the invention, the electron donor comprises indophynol and/or ascorbate.

According to an aspect of some embodiments of the present invention there is provided a nanostructure comprising at least one inorganic nanoparticle bound to a photocatalytic unit of a photosynthetic organism, the inorganic nanoparticle being a metal nanoparticle or a nanoshell.

According to some embodiments of the invention, the at least one inorganic nanoparticle binds to a polypeptide of a reaction center of the photocatalytic unit.

According to some embodiments of the invention, the at least one inorganic nanoparticle binds to an antenna chlorophyll of the photocatalytic unit.

According to some embodiments of the invention, the inorganic nanoparticle is attached to an electron acceptor side of the reaction center.

According to some embodiments of the invention, the inorganic nanoparticle is attached to an electron donor side of the reaction center.

According to some embodiments of the invention, the inorganic nanoparticle is attached to the photocatalytic unit via a bifunctional connecting molecule.

According to some embodiments of the invention, a diameter of the at least one inorganic nanoparticle is about 1 nm to 50 nm.

According to some embodiments of the invention, a diameter of the at least one inorganic nanoparticle is about 21 nm.

According to some embodiments of the invention, a length of the bifunctional connecting molecule is about 1.5 nm.

According to some embodiments of the invention, the inorganic nanoparticle is generally spherical.

According to some embodiments of the invention, the metal nanoparticle comprises silver or gold.

According to an aspect of some embodiments of the present invention there is provided a device comprising a nanostructure attached to at least one electrode, the nanostructure comprising at least one inorganic nanoparticle bound to a photocatalytic unit of a photosynthetic organism, the inorganic nanoparticle being a metal nanoparticle or a nanoshell.

According to some embodiments of the invention, the device serves as a component selected from the group consisting of a photodiode, a phototransistor, a logic gate, a solar cell and an optocoupler.

According to some embodiments of the invention, the electrode comprises a transition metal.

According to some embodiments of the invention, the transition metal is selected from the group consisting of silver, gold, copper, platinum, nickel, aluminum and palladium.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph illustrating the composition of elements in a platinized PS I monolayer, prepared according to some embodiments of the present invention. Surface composition was analyzed by XPS of platinized PS I monolayer on a gold slide deposited on a silicon wafer.

FIG. 2 is a schematic presentation of energy levels in a PS I in junction with gold and platinum.

FIG. 3 is a graph illustrating the cyclic voltametry of measurement of platinized PS I monolayer, prepared according to some embodiments of the present invention. The electrochemical measurements set-up included an Ag/AgCl/1M KCl reference electrode, a Pt counter electrode and a working electrode made from a PS I monolayer on a gold surface. The cell medium contained 50 mM tris-Cl, pH 7 and 50 μM methyl viologen that mediated electrons between PS I and the Pt electrode under N₂ atmosphere. The measurements were conducted in the dark (black line) and under illumination (red line) with an incandescence light at intensity of 26.5 mW/cm².

FIGS. 4A-C are illustrations prepared by a computer simulation technique, showing the molecular structure of the platinized PS I of the present embodiments and their multilayer coverage of a gold surface. (FIG. 4A) Light-induced charge separation (arrow) across the electron transport chain (rods, purple and space fill) showing chlorophyll and carotenoid molecules (rods, green and orange) in PS I modeled as polypeptide back-boned structure (cyano) with cysteine mutants Y634C shown in space fill, yellow. Pt ion (dots) bound to PS I is reduced to Pt (space fill, dark gray) by electrons from the terminal iron sulphur cluster (space fill). The electron transport chain in PS I contains a special pair of chlorophyll a (P700) that transfers electrons following photo excitation in 1 picoseconds (ps) to a monomeric chlorophyll a (Chl), through two intermediate phylloquinones (PQ) to the final acceptors: three [4Fe-4S] iron sulfur centers (FeS) that are reduced in 0.2 μs [P. R. Chitnis, N. Nelson, in Photosynthetic Apparatus: Molecular Biology and Operation, Vol. 7B (Eds: L. Bogoras, I. K. Vsil), Academic Press, N.Y. 1991, 177]. The redox potential of the primary donor P700 is +0.49 V and that of the final acceptor FeS is −0.53 V. (FIG. 4B) A schematic presentation of Pt crystal deposited on a PS I molecule (space fill model). (FIG. 4C) A schematic presentation of a PS I multilayers (space fill model) on gold surface. Atom color codes are: C gray, O red, N blue and S yellow and Pt dark gray. The images were modeled by PyMole software from the coordinates in PDB 1JBO file.

FIGS. 5A-F are scanning probe microscopy images of a platinized PS I monolayer prepared according to some embodiments of the present invention. Topographic 3D images of PS I (FIG. 5A) and of the platinized PS I (FIG. 5B) monolayers obtained by AFM. Phase contrast 3D images of PS I (FIG. 5C) and platinized PS I (FIG. 5D), proteinase K digested PS I (FIG. 5E) and platinized PS I (FIG. 5F) monolayers. In the phase contrast measurements, features of the protein can be seen under the metal surfaces of the platinized PS I.

FIGS. 6A-C are Kelvin probe microscopy images of PS I mono- and bi- and tri-layers, prepared according to some embodiments of the present invention. (FIG. 6A) Light-induced surface potential differences of 3D images PS I bi-layer. (FIG. 6B) Kinetic recording of reversible light induced (shutter off time 0.7 ms) photo-potential of mono- (blue), bi- (red) and tri-layers (green) of PS I are shown. (FIG. 6C) Light intensity dependence of the photo-potential of PS I mono- (black) and tri-layers (red). Surface potential measurements were done by KPFM. Illumination was provided by a diode laser with maximum power output of 40 mW at 670 nm.

FIG. 7 is an illustration prepared by a computer simulation technique showing the molecular structure of a self assembled multilayer of platinized PSI, prepared according to some embodiments of the present invention. Atom color codes are: C gray, O red, N blue and S yellow and Pt dark gray. Pt crystal of 2 nm deposited on a PS I molecule (space fill model) serve as junctions connecting sequential layers by formation of Pt-sulfide bond between the cysteines of PSI and the Pt junctions. The images of the coordinates were modeled by PyMole software from the coordinates in PDB 1JBO file.

FIG. 8 is a graph illustrating the absorption spectra of self assembled multilayers, prepared according to some embodiments of the present invention. The absorption spectrum of a platinized self assembled monolayer on gold surface (red) that serves as a template for fabrication of multilayers (black) self assembled from a suspension of platinized PSI. An estimated 25 layers are assembled.

FIGS. 9A-B are schematic illustrations of a photoactive nanostructure which comprises a nanoparticle (NP) bound to a photocatalytic unit. FIGS. 9A-B illustrates a configuration (FIG. 9A) and geometry (FIG. 9B) of the photoactive nanostructure in an embodiment of the invention in which the solid nanoparticle is a metal nanoparticle and the photocatalytic unit is a photosystem I (PS I) reaction center (RC).

FIG. 10 is a graph illustrating the calculated enhancement factors for the photoactive nanostructure as a function of the wavelength, in embodiments of the invention in which the solid nanoparticle is Au NP and in embodiments of the invention in which the solid nanoparticle is a Ag NP.

FIGS. 11A-B are graphs illustrating the calculated enhancement factors for the photoactive nanostructure as a function of the wavelength, in embodiments of the invention in which the solid nanoparticle is a Au nanoshell and in embodiments of the invention in which the solid nanoparticle is a Ag nanoshell.

FIG. 12A is a graph illustrating the calculated enhancement energy transfer time for Au and Ag nanoshells, according to various exemplary embodiments of the present invention.

FIG. 12B is a graph illustrating the quantum yield for Au and Ag nanoshells, according to various exemplary embodiments of the present invention.

FIG. 12C is a graph illustrating the relative rate of quinine production for Au and Ag nanoshells, according to various exemplary embodiments of the present invention.

FIG. 13 is a graph illustrating the calculated rate of quinine production for a single AgNP conjugated with a reaction center, according to various exemplary embodiments of the present invention. Inset: quantum yield for the same system.

FIGS. 14A-B are transmission electron microscopy images of ˜5 nm AuNP/PSI hybrid photoactive nanostructure (FIG. 14A) and AgNP/PSI hybrid photoactive nanostructure (FIG. 14B), prepared according to some embodiments of the present invention. The bar is 10 nm for FIGS. 14A and 20 nm for FIG. 14B.

FIG. 15 is a graph illustrating the Plasmon enhancement of PSI absorption spectra of the photoactive nanostructure in embodiments of the invention in which the solid nanoparticle is a gold nanoparticle and the photocatalytic unit is a photosystem I (PS I) reaction center. The absorption spectrum of PSI in solution (blue) is enhanced by plasmon resonance on the formation of ˜5 nm AuNP/PSI hybrids (black). Addition of thioglycolate the partially dissociates the PSI metal NP bond decreases the enhancement (red). The broad absorption band of the AuNP plasmons (maxima at 550 nm) is seen.

FIG. 16 is a graph illustrating the Plasmon enhancement of PSI absorption spectra of the photoactive nanostructure in embodiments of the invention in which the solid nanoparticle is a silver nanoparticle and the photocatalytic unit is a photosystem I (PS I) reaction center. Plasmon enhancement of PSI absorption spectra in silver nanoparticles/PSI hybrids. The absorption spectrum of PSI in solution (blue) is enhanced by plasmon resonance on the formation of ˜5 nm AuNP/PSI hybrids (black). Addition of thioglycolate the partially dissociates the PSI metal NP bond decreases the enhancement (red).

FIG. 17 is a graph illustrating the Plasmon enhancement of the circular dichroism (CD) specrta of the photoactive nanostructure in embodiments of the invention in which the solid nanoparticle is a gold or silver nanoparticle and the photocatalytic unit is a photosystem I (PS I) reaction center. Plasmon enhancement of PSI CD spectra (black) in ˜5 nm AuNP/PSI (red) and AgNP/PSI (blue) hybrids is shown. Addition of thioglycolate partially dissociates the PSI metal NP bond and decreases the enhancement in AuNP/PSI (red dots) and AgNP/PSI (blue dots).

FIG. 18A is a schematic diagram illustrating the geometry of an exemplary hybrid photoactive nanostructure of some embodiments of the present invention. Förster energy transfer couples the CdTe nanoparticle and reaction center.

FIG. 18B is a schematic illustration of the physical process of an exemplary hybrid photoactive nanostructure of some embodiments the present invention. Arrows show the most important physical processes, including the NP-RC energy transfer, energy relaxation, and electron-hole separation.

FIG. 19 is a graph of absorption cross section of the reaction center and the spectrum of exciton emission of a semiconducting nanoparticle. Inset: Absorption cross sections for the reaction center and nanoparticle with a radius R_{NP}=4 nm.

FIG. 20 is a graph illustrating the calculated rates of generation of excited electrons for the RC alone and for the hybrid RC-NP photoactive nanostructure of the present embodiments. The light intensity is I=1 W/cm². The red curve corresponds to the spectral energy density U_{λ} of sunlight radiation as a function of the wavelength λ, according to the expression

$${U}_{λ} ∝ {λ}^{- 5} / ( {e}^{\frac{2 π \cdot ℏ c}{λ \cdot {k}_{B} T}} - 1 ) .$$

Inset: The ratio of the rates for the RC and hybrid complex.

FIG. 21 is a graph of the calculated ratio of integrated rate for the RC to the integrated rate of the hybrid RC-NP nanostructure of the present embodiments as a function of the bio-linker length. Inset: Quantum yields for the RC and hybrid nanostructure as functions of the bio-linker length.

FIG. 22 is a schematic illustrating the geometry for photocurrent experiments according to some embodiments of the present invention. The FT mechanism couples three semiconductor NPs and a RC.

FIG. 23 is a schematic illustration of an optoelectronic device, according to various exemplary embodiments of the present invention.

FIG. 24 is schematic illustration of a photodiode device, according to various exemplary embodiments of the present invention.

FIG. 25 is a schematic illustration of a phototransistor, according to various exemplary embodiments of the present invention.

FIG. 26 is a simplified illustration of an optocoupler, according to various exemplary embodiments of the present invention.

FIGS. 27A-B are simplified illustrations of an optoelectronic device, according to various exemplary embodiments of the present invention.

FIG. 28 illustrates an energy-level diagram in an embodiment in which one electrode of the device is made of aluminum and another electrode is made of indium tin oxide.

FIGS. 29A-B are schematic illustrations of an optoelectronic array, according to various exemplary embodiments of the present invention.

FIG. 30 is a flowchart diagram of a method suitable for fabricating an optoelectronic device, according to various exemplary embodiments of the present invention

FIGS. 31A-D are schematic process illustrations of various method for fabricating the optoelectronic device, according to various exemplary embodiments of the present invention.

FIGS. 32A-D are schematic process illustrations of various method steps for fabricating the optoelectronic array, according to various exemplary embodiments of the present invention.

### DESCRIPTION OF SPECIFIC EMBODIMENTS OF THE INVENTION

The present invention, in some embodiments thereof, relates to a photoactive nanostructure comprising one or more solid nanoparticles bound to a photocatalytic unit of a photosynthetic organism. The present invention also relates to fabrication of devices with multi-layers of photocatalytic units.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Photosynthesis is the biological process that converts electromagnetic energy into chemical energy through light and dark reactions. In oxygenic plants and cyanobacteria, photon capture and conversion of light energy into chemical energy take place in specialized membranes called thylakoids. In higher plants, the thylakoids are located in the chloroplast.

Using intricate mathematical analysis, the present inventors have shown that the rate of optical generation of charge separation inside a photosynthetic protein can be greatly increased by conjugation with a conductive or semiconductor nanoparticle. The enhancement effect comes from larger optical absorption cross-section of, and/or higher excitation rate in, the conjugated nanostructure compared to the photoactive photosynthetic protein in the absence of inorganic nanoparticles therein.

For example, some embodiments of the present invention comprise a photoactive nanostructure comprising one or more solid semiconductor nanoparticles bound to a photocatalytic unit of a photosynthetic organism. The semiconductor nanoparticle of the present embodiments may exhibit a number of unique optical properties due to quantum confinement and surface energy effects. The semiconductor nanoparticle of the present embodiments absorbs light with absorption cross section which is significantly higher (preferably at least 5-10 times higher) than the absorption cross section of the photocatalytic unit to which it is bound. Excitons, generated in the semiconductor nanoparticle in response to photons absorbed thereby, are transferred to the photocatalytic unit via Förster energy transfer. The exiton transferred from the semiconductor nanoparticle to the photocatalytic unit is efficiently trapped because the induced charge separation, (electron hole separation that efficiently drives electron transfer), is faster than the energy transfer rate between the nanoparticles and the photosystem.

The present inventors analyzed a model nanostructure comprising PSI attached to a semiconductor nanoparticle made of CdTe with a radius of 4 nm. This nanoparticle generates exciton emission at 677 nm which matches the absorption maxima of PS I and therefore can be used for light-harvesting applications, such as, but not limited to, the absorption maxima although all wavelength between 400 nm and 700 nm are contemplated. Because the absorption cross section of the nanoparticle (NP) is 100 fold larger than that of PS I, the rate of electron transport generation in the hybrid photoactive nanostructure is calculated to be increased by 77 fold compared to that of PS I. The present inventors rationalized that NPs made of other semiconductor materials can be selected by tuning their size in relation to their absorption band gap energy to generate excitons emission in the wavelength that is efficiently absorbed by PS I.

Some embodiments of the present invention provide a photoactive nanostructure comprising one or more solid conductive nanoparticles (e.g., metallic nanoparticle) bound to a photocatalytic unit of a photosynthetic organism. In these embodiments, resonant collective oscillations of conduction electrons, also known as plasmons, are excited within conductive nanoparticle by the optical field. The resonance frequency of the plasmon depends on the properties of the nanoparticles, particularly the dielectric function and geometry, but may also depend on the surrounding medium, which according to various exemplary embodiments of the present invention is a photocatalytic unit. The resonance leads to a spectrally selective light absorption and an enhancement of the local field confined on and close to the surface of the conductive nanoparticle. This enhancement increases the probability of photons to generate excitons in the photocatalytic unit via light absorption. The excitons are efficiently dissociated within the photocatalytic unit.

The present inventors have demonstrated that a conductive nanoparticle of a hybrid nanostructure which comprises a conducting nanoparticle and a photosynthetic protein, can be selected to generate plasmon with energy that can efficiently enhance absorption by the chlorophylls and enhance the photocurrent response in the photosynthetic protein.

The type of metal used and the size of the NP can be tuned to generate plasmon with energy that can be efficiently absorbed by PS I. For example the present inventors calculated that silicon coated gold and silver NP of 21 nm in diameter can generate plasmon at wavelengths that enhance the absorption by PS I with peak missions at about 700 nm. Plasmons of such energy can efficiently enhance the absorption by the two absorption maxima of PS I. The present inventors calculated that light energy can enhance electron generation in the PS I hybrid gold NP and silver NP by factors of 10 and 15 fold, respectively at the peak emission. By tuning the total size and the size of the coated layer other metals can be used to generate plasmons with energy that can efficiently enhance the absorption by the pigments and enhance the efficient charge separation process and the current generated by PS I in future optoelectronic devices.

Whilst reducing the present invention to practice, the present inventors succeeded in combining PSI with various inorganic nanostructures, including, without limitation, conductive (e.g., metal) nanoparticles, semiconductor nanoparticles, conductive nanoshells, semiconductor nanoshells and the like. The present inventors have discovered that fabrication of hybrid structures which include PSI and a conductive or semiconductor nanostructure can be utilized to enhance light energy conversion in optoelectronic devices.

Whilst further reducing the present invention to practice, the present inventors have devised a method of fabricating serially-oriented multilayers of PSI. The fabrication is mediated by the photo-catalytic specificity that reduces metal ions to nanometric metal patches on the reducing side of PS I forming junctions with the oxidizing end of the proteins through metal-sulfide bond of genetically-engineered cysteine mutants. The platinized monolayer serves as a template on which serially oriented multilayers are self assembled by incubation with a suspension of platinized PSI. The dry hybrid nanoparticles and multilayers in hybrid bio-solid-state electronic devices increase photo-voltage and photo-current, resulting from the larger absorption cross-section and the serial-arrangement of PS I.

Thus, according to one aspect of some embodiments of the present invention, there is provided a nanostructure comprising a photocatalytic unit of a photosynthetic organism attached to at least one inorganic nanoparticle.

As used herein the term “nanostructure” refers to a structure on the sub-micrometer scale which includes one or more particles, each being on the nanometer or sub-nanometer scale and commonly abbreviated “nanoparticle”. The distance between different elements (e.g., nanoparticles, molecules) of the structure can be of order of several tens of picometers or less, or between several hundreds of picometers to several hundreds of nanometers. Thus, the nanostructure of the present embodiments can comprise a nanoparticle, an arrangement of nanoparticles, or any arrangement of one or more nanoparticles and one or more molecules.

As used herein, the phrase “photocatalytic unit” refers to a complex of at least one polypeptide and other small molecules (e.g. chlorophyll and pigment molecules), which when integrated together work as a functional unit converting light energy to chemical energy. As mentioned herein above, the photocatalytic units of the present embodiments are present in photosynthetic organisms (i.e. organisms that convert light energy into chemical energy). Examples of photosynthetic organisms include, but are not limited to green plants, cyanobacteria, red algae, purple and green bacteria.

Thus, examples of photocatalytic units which can be used in accordance some embodiments of the present invention include biological photocatalytic units such as PS I and PS II, bacterial light-sensitive proteins, bacterial light-sensitive proteins, bacteriorhodopsin, photocatalytic microorganisms, pigments (e.g., proflavine and rhodopsin) and algae. Preferably, the photocatalytic unit of the present embodiments is photosystem I (PS I). According to one embodiment, the photocatalytic unit is not comprised in a dye.

PS I is a protein-chlorophyll complex, present in green plants and cyanobacteria, that is part of the photosynthetic machinery within the thylakoid membrane. It is ellipsoidal in shape and has dimensions of about 9 by 15 nanometers.

The PS I complex typically comprises chlorophyll molecules which serve as antennae which absorb photons and transfer the photon energy to the reaction center. The reaction center is responsible to capturing this energy and utilizing it to drive photochemical reactions. In the reaction center, an electron is released from P700 and transferred to a terminal acceptor at the reducing end of PSI through intermediate acceptors, and the electron is transported across the thylakoid membrane.

It will be appreciated that PS Is derived from cyanobacteria differ from those derived from plant and bacterial reaction centers. This is due to the fact that all chlorophyll molecules and carotenoids are integrated into the core subunit complexes in cyanobacteria while in plant and other bacterial reaction centers the antenna chlorophylls are bound to chlorophyll-protein complexes that are attached to the core subunits.

The PS I reaction center from cyanobacteria (e.g. from Synechocystis sp. PCC6803) consists of 12 polypeptides, some of which bind 96 light-harvesting chlorophyll and 22 beta carotenoid molecules. The electron transport chain contain P700, A₀, A₁, Fₓ, F_{A} and F_{B} representing a chlorophyll a dimmer, a monomeric chlorophyll a, two phylloquinones and three [4Fe-4S] iron sulfur centers, respectively. The reaction center core complex is made up of the heterodimeric PsaA and PsaB subunits, containing the primary electron donor, P700, which undergoes light-induced charge separation and transfers an electron through the sequential carriers A₀, A₁ and Fₓ. The final acceptors F_{A} and F_{B} are located on another subunit, PsaC.

Methods of isolating photocatalytic units from plants and bacteria are known in the art. An exemplary method for removing photocatalytic units from photosynthetic organisms is described in Example 1 of the Examples section hereinbelow. The present embodiments also envisage using any other methods of purification and isolation so long as the photocatalytic unit remains functional. The photocatalytic units may be isolated as polymers e.g. trimers or as single monomers. The photocatalytic units may be fully isolated or part of a membrane preparation. Methods of preparing membrane extracts are well known in the art. For example, Qoronfleh et al., [J Biomed Biotechnol. 2003; 2003(4): 249-255] teach a method for selective enrichment of membrane proteins by partition phase separation. Various kits are also commercially available for the preparation of membrane extracts such as from Sigma-Aldrich (ProteoPrep™ Membrane Extraction Kit).

As used herein, the term “nanoparticle” refers to a particle or particles having an intermediate size between individual atoms and macroscopic bulk solids. Generally, nanoparticle has a characteristic size (e.g., diameter for generally spherical nanoparticles, or length for generally elongated nanoparticles) in the sub-micrometer range, e.g., from about 1 nm to about 500 nm, or from about 1 nm to about 200 nm, or of the order of 10 nm, e.g., from about 1 nm to about 100 nm. In some embodiments of the present invention the characteristic size is from about 1 nm to about 20 nm. The nanoparticles may be of any shape, including, without limitation, elongated particle shapes, such as nanowires, or irregular shapes, in addition to more regular shapes, such as generally spherical, hexagonal and cubic nanoparticles. Additionally, the nanoparticles may be single-crystalline, polycrystalline or amorphous in nature. A plurality of nanoparticles may include nanoparticles of a single type of crystallinity or may consist of a range or mixture of crystallinity (i.e., some particles crystalline, others amorphous). According to one embodiment, the nanoparticles are generally spherical.

According to one embodiment, the nanoparticle is a semiconductor nanoparticle. In some embodiments, the semiconductor nanoparticles have a size on the order of the Bohr exciton radius, or the de Broglie wavelength, which allows individual semiconductor nanoparticles to trap individual or discrete numbers of charge carriers (either electrons or holes) or excitons, within the nanoparticle.

The diameter of semiconductor nanoparticles which are envisioned by the present embodiments are typically between 1 nm to 50 nm, e.g. between 2 nm to 20 nm. According to one embodiment the diameter of a CdTe nanoparticle is about 8 nm.

Exemplary semiconductor materials which may be used to fabricate the semiconductor nanoparticles of the present embodiments include, but are not limited to Cdte, CdS, CdSe GaAs, Si, Ge, GeN, SiGe, AlGaAs, InGaAs, InGaP, AlInP, GaInAsP, GaN, AlGaN, and the like.

According to another embodiment, the nanoparticle is an electrically conductive nanoparticle, such as a metal nanoparticle or a nanoshell.

The electrically conductive nanoparticles of the present embodiments typically have a diameter of about 1 nm to 50 nm. For example, the diameter of a silver or gold nanoparticle may be about 21 nm.

The structure size and shape of the electrically conductive nanoparticles of the present embodiments can be selected in accordance with the spectrum of light which the photoactive nanostructure is designed to absorb. For example, the characteristic size (e.g., diameter for generally spherical nanoparticles, or length for generally elongated nanoparticles) of the nanoparticles can be selected such that the resonance frequency of the nanoparticles and the frequency of the impinging light coincide. In embodiments in which the photoactive nanostructure absorbs light in the visible range (e.g., 400-800 nm), the characteristic size of the electrically conductive nanoparticles can be from about 1 nm to about 50 nm.

Suitable metals for forming the metallic nanoparticles include the noble and coinage metals, but other electrically conductive metals may also be employed. Metals that are particularly well suited for use in the nanoparticles include but are not limited to gold, silver, copper, palladium, lead, iron or the like. Gold and silver are preferred. Alloys or non-homogenous mixtures of such metals may also be used.

According to one embodiment, the metallic nanoparticle does not comprise platinum.

It is often desired to further minimize the nanoparticles size, for example, to increase the effect of optical field enhancement. This may be done, by providing nanoparticles which include a dielectric core and a conducting shell layer. Such nanoparticles are referred to herein as “nanoshells”. For any given core and shell materials, the ratio between the core radius and the total radius of nanoshells can be chosen for providing optical field enhancement. In various exemplary embodiments of the invention the radii ratio is selected so as to increase or maximize scattering and reduce or minimize absorption at a specific resonance frequency. Based on the core to total radii ratios, the nanoshells of the present embodiments can manifest plasmon resonances at any wavelength from ultraviolet to infrared. Core diameters suitable for the present embodiment are from about 5 nm to about 20 nm, and the shell diameter suitable for the present embodiment are from about 5 nm to about 30 nm.

Suitable metals for forming the outer layer of the nanoshells include the noble and coinage metals, but other electrically conductive metals may also be employed. Metals that are particularly well suited for use in shells include but are not limited to gold, silver, copper, palladium, lead, iron or the like. Gold and silver are preferred. Alloys or non-homogenous mixtures of such metals may also be used.

The process of manufacturing nanoshells having a dielectric core and a conducting shell, is known in the art and is described, for example, in international Patent Publication Nos. WO 01/06257 and WO 02/28552, the contents of which are hereby incorporated by reference.

The dielectric core of the nanoshell of the present embodiments can be, for example, a semiconductor material (e.g. silicon), an organic molecule, an organic super-molecular structure, or any mixture of non-conductive materials.

The thickness of the coating on the nanoshell can vary between 1 nm and 15 nm depending on the coating metal and the core material.

The structure size and shape of the nanoparticles can be designed in accordance with the specific application for which system they are used. For example, in embodiments in which the nanoparticle is electrically conductive nanoparticle, the size of the nanoparticle can be selected so as to generate plasmon with energy that enhances efficiently the absorption of photons by the photocatalytic unit. In embodiments in which the nanoparticle is a semiconductor nanoparticle, the size and/or type of the semiconductor nanoparticle can be selected according to the absorption band gap energy required to generate exciton emission in a wavelength that is efficiently absorbed by the photocatalytic unit.

As mentioned, the nanostructures of the present embodiments are constructed such that the nanoparticles are attached (i.e. bound) to a photocatalytic unit.

Any binding is envisaged according to the present embodiments so long as the binding allows the photocatalytic unit to retain activity. Such binding includes direct binding (e.g. the polypeptides in photocatalytic units may comprise or may be genetically modified such that they comprise functional groups for covalent binding to a nanoparticle) or indirect binding (e.g. via a bifunctional connecting molecule that has one functional group bound to the photocatalytic unit and one functional group bound to the nanoparticle). The present embodiments also envisage non-covalent binding between the photocatalytic unit and the nanoparticle. Non-covalent binding can be effected, for example, via electrostatic (ionic) interactions, hydrophobic interactions, hydrogen bonds and physical interactions such as, for example, absorbance, entrapment, swelling, adherence and the like. For example, a nanoparticle may be coated with organic molecules and the photocatalytic unit may be non-covalently adsorbed—see for example Lee et al., [J. Phys. Chem. B 2000].

When the nanoparticle is comprised of a semiconductor, the binding and the semiconductor nanoparticle are preferably selected such that transfer of electrons from the photocatalytic unit to the semiconductor nanoparticle is prevented, or at least suppressed relative to transfer of excitons from the semiconductor nanoparticle to the photocatalytic unit. This is in sharp contrast to Weng et al [abstract of Acta Botanica Sinica, 2003, 45(4) 488-493] who teaches electron transfer between the pigments in a light harvesting 2 complex and semiconductor nanoparticles.

As used herein, a process A is suppressed relative to a process B, if the characteristic time constant of process A is at least one, more preferably at least two, e.g., three orders of magnitude longer than the characteristic time constant of process B.

Quantum mechanically, a shorter characteristic time constant corresponds to higher transition probability between states. Thus, when the time constant characterizing transfer of excitons from the semiconductor nanoparticle to the photocatalytic unit is significantly shorter than the time constant characterizing transfer of electrons from the photocatalytic unit to the semiconductor nanoparticle, the probability for exciton transfer is higher than the probability for electron transfer. In some embodiments of the present invention an electron transfer from the photocatalytic unit to the semiconductor nanoparticle occurs in a time scale of the order of microseconds and transfer of excitons occurs in a time scale of the order of nanoseconds.

Thus, unlike traditional constructions in which the dominant mechanism is electron transfer, the nanostructure of the present embodiments facilitates transfer of exciton via Förster energy transfer

According to some embodiments of the present invention, the PSIs retain photocatalytic activity following attachment to a solid surface.

Herein, the phrase “photocatalytic activity” refers to the conversion of light energy to chemical energy. Preferably, the photocatalytic units retain at least 20%, more preferably at least 30%, more preferably at least 40%, more preferably at least 50%, more preferably at least 60%, more preferably at least 70%, more preferably at least 80%, more preferably at least 90%, e.g., about 100% the activity of the photocatalytic unit prior to attachment to the nanoparticle.

The nanoparticle of the present embodiments may bind to a polypeptide of the reaction center of the photocatalytic unit, (either to the electron acceptor side or the electron donor side). Alternatively, or additionally, the nanoparticle of the present embodiments may bind to antennae chlorophyll of the photocatalytic unit.

According to an embodiment of the present invention, the nanoparticle binds directly to a modified polypeptide of the reaction center of the photocatalytic unit.

As used herein, the phrase “modified polypeptide” refers to a polypeptide comprising a modification as compared to the wild-type polypeptide. Typically, the modification is an amino acid modification. Any modification to the sequence is envisaged according to the present embodiments so long as the polypeptide is capable of covalent attachment to the nanoparticle and the photocatalytic unit retains a photocatalytic activity. Examples of modifications include a deletion, an insertion, a substitution and a biologically active polypeptide fragment thereof. Insertions or deletions are typically in the range of about 1 to 5 amino acids.

The site of modification is selected according to the suggested 3D structure of the photocatalytic unit. Evidence relating to the 3D structure of photocatalytic units may be derived from X-ray crystallography studies or using protein modeling software. The crystalline structure of PS I from Thermosynechococcus elongatus and from plants chloroplast has been resolved to 2.5 Å at 4.4 Å, respectively [P. Jordan, et al., Nature 2001, 411 909-917; A. Ben Shem, F. Frolow, N. Nelson, Nature 2003, 426 630-635].

The amino acid to be replaced or the site of insertion is typically on the external surface of the photocatalytic unit (e.g. on an extra membrane loop). Preferably, the amino acids to be replaced or the site of insertion is in a position which does not cause steric hindrance. Also it is preferred that the mutations are positioned near the P700 of the photocatalytic unit to secure close proximity between the reaction center and the solid surface in order to facilitate an efficient electric junction.

According to some embodiments of the present invention, the modification is a substitution (i.e. replacement) comprising a functional group side chain which is capable of mediating binding to a metal surface, e.g an amino acid that comprises a thiol group such as a cysteine. Particularly preferred coordinates for mutation of PS I from Synechocystis sp. PCC 6803 in PsaB include single mutations D236C, S247C, D480C, S500C, S600C and Y635C or double mutations D236C/Y635C and S247C/Y635C. In PsaC, a particularly preferred site for a mutation is W32C. In addition, a triple mutation may be generated in the photocatalytic units (e.g. PsaC//PsaB W32C//D236C/Y635C).

In a particular embodiment of the present invention, the photocatalytic units of the present embodiments comprise polypeptides as set forth by SEQ ID NOs: 1-10.

For methods of generating the modified polypeptides of this embodiment of the present invention please refer to International Patent Publication No. WO2006/090381, the entire contents of which are incorporated herein by reference.

The modified photocatalytic unit of the present embodiments can be covalently attached to nanoparticles by directly reacting the substituting residue with a hydrophilic surface of a solid substrate. For example, where the substituting residue is cysteine, the attachment can be done by incubating the modified photocatalytic unit with gold or other metals nanoparticles for a period sufficient to form a sulfide bond. Other attachment methods are also contemplated.

As mentioned, the present embodiments also contemplate non-direct binding of the nanoparticles to the photocatalytic unit via a bifunctional connecting molecule.

The bifunctional connecting molecule may be attached to a free carboxyl of the photocatalytic unit, a free primary amine of the photocatalytic unit and/or to a thiol group in the photocatalytic unit.

The length of the bifunctional connecting molecule may be selected according to the moiety that functions in binding to the solid surface on one hand and a functional group that binds to the protein.

A typical length of a bifunctional connecting molecule is typically between 0.5 nm and 6 nm (e.g. 1.5 nm).

According to one embodiment the bifunctional connecting molecule comprises a succinylimide moiety.

For indirect binding of a semiconductor (e.g. silicon) nanoparticle to a photocatalytic unit, the silicon surface may be modified by chemisorption of silan amine.

The free amine groups may then be covalently bound to the free carboxyls of the surface of PSI by carbodiimide chemistry. For example, reaction in aqueous solution pH 7, containing 1-Ethyl-3-[3-dimethylaminopropyl]carbodiimide hydrochloride (EDC or EDAC) connects the free amines on the surface of the silicon with the carboxyls of the PSI protein when laid on top of the modified semiconductor surface. Alternatively, the free amine groups of silane amine may be covalently bound to the free primary amines of the protein by a short connecting molecule Sulfo-MBS (m-Maleimidobenzoil-N-hydroxysulfosuccinimide ester) in which the succinimide is first bound to the silanamine at the surface to form an oriented monolayer. Next, the maleinide end of the molecule binds (at pH 8) the free amines of the photocatalytic unit.

The semiconductor nanoparticle can be prepared for the attachment of the modified photocatalytic unit using the following procedure which is not intended to be limiting. The semiconductor nanoparticle can be cleaned and etched. Following rinsing, the etched semiconductor nanoparticle can be immediately immersed in a solution selected to facilitate chemical adsorption. For example, the solution can comprise ECMA, BMPA or the like which can be chemisorbed to the etched surface through their carboxyl end to form a self-assembled monolayer on the surface. An aqueous solution can be used for terminating the chemisoption.

The surface can be hydroxylated and then coated with amino silan using a reagent such as, but not limited to, (3-aminopropyl)-Diethoxymethylsilane or (3-aminopropyl) ethoxydimethylsilane. Linker molecules, e.g., m-Maleimidobenzoyl-N-hydroxydulfosuccinimide ester can then be attached to the amino silan.

For indirect binding of a metal nanoparticle to a photocatalytic unit, bifunctional short connecting molecules containing thiol can connect the NP to metal surfaces by formation of sulfide bonds.

It will be appreciated that the nanostructures of the present embodiments may comprise more than one nanoparticle. Various arrangements of nanoparticles are contemplated by the present inventors including direct attachment of more than one nanoparticle to the photocatalytic unit (e.g. one on the acceptor side and one on the donor side of the reaction center, or one attached to a polypeptide of the reaction center and one attached to the antennae chlorophyll) or attachment of a second nanoparticle to a first nanoparticle which is itself bound to the photocatalytic unit.

Following generation, the nanostructures of the present embodiments may be attached to a solid surface (e.g. electrode) which is preferably of macroscopic size, e.g., having a surface area of at least 1 mm² so as to fabricate a device. The solid surface is preferably of macroscopic size, e.g., having a surface area of at least 1 mm². Such a device may serve as a component in, e.g., a photodiode, a phototransistor, a logic gate, a solar cell, an optocoupler and the like. The attachment to solid surface can be by covalent or non-covalent bonding (electrostatic).

The solid surface is preferably an electrically conductive material, such as a transition metal. Examples of transition metals which may be used according to the present embodiments include, but are not limited to silver, gold, copper, platinum, nickel, alluminum and palladium.

According to some embodiments of the present invention, the modified photocatalytic unit retains photocatalytic activity following attachment of the hybrid nanostructure to a solid surface.

Any method is contemplated for attaching the nanostructures of the present embodiments to a solid surface, provided the photocatalytic unit retains its activity.

The nanostructures of the present embodiments are preferably attached in an oriented manner to the solid support. Such construction is advantageous because it prevents the nanostructures from neutralize each others charge. This facilitates an overall photocatalytic activity of the nanostructures on the solid support.

According to one embodiment, when the photocatalytic unit comprises a cysteine substitution, the cysteine may be reacted with a fresh, clean, metal surface to form a metal-sulfide bond. Flat metal surfaces may be prepared by evaporation of 200 nm metal on glass or silicon wafers. These surfaces are annealed at 350° C. for 1 hour under vacuum and etched if required. Typically, the excess protein is washed and the self assembled oriented monolayer of photocatalytic units is dried. In this arrangement, the nanostructures of the present embodiments are preferably attached to the photocatalytic unit via a bifuctional molecule. For example, a succilimide moiety may be attached to the free amines of the photocatalytic unit and a thiol to a metal nanostructure.

Alternatively, the nanostructures of the present embodiments may be attached to a solid support via bifunctional aromatic dithiols. In this arrangement, the photocatalytic unit may be attached to the nanostructure through the thiols of the cysteine mutants.

Methods of measuring photocatalytic activity on surfaces fabricated therewith include measuring the photovoltage properties of the fabricated surfaces. The photovoltage properties may be measured for example by Kelvin probe force microscopy (KPFM).

It will be appreciated that the nanostructures of the present embodiments may be attached to the solid support as a monolayer or a multilayer. Multilayers are preferred when hybrid bio-solid-state electronic devices in which an increase in photo-voltage are required.

Fabrication of serially-oriented multilayers of photosynthetic reaction center photosystem I (PS I) may be mediated by the photo-catalytic specificity that reduced metal ions to nanometric metal patches on the reducing side of PS I forming junctions with the oxidizing end of the proteins through metal-sulfide bond of genetically-engineered cysteine mutants. Specific methods of generating multilayers of photosystem Is to fabricate a device are described in Example 1 and 2, herein below. The Pt atoms are typically attached to the photocatalytic unit at the opposite side of the metal/semiconductor surface.

Both methods utilize metal bonding by photoreducing Pt⁴⁺ ions in solution by the PS I monolayer. Pt⁴⁺ ions can be photoreduced by PS I monolayer at the reducing end of the protein and Pt is deposited. Such monolayer is characterized by metal deposition on top of each of the PS I as the phase angle increases with the stiffness of the substrate. Using such technique, several oriented monolayers can be formed on top of each other, where the Pt—S bond connects between adjacent monolayers.

In Example 1, PSIs are first connected to a solid surface so as to generate an orientated monolayer. The monolayer is platinized and a second layer of PSIs is then added. Sequential rounds of platinization and addition of PSIs results in generation of multilayers of PSI. Platinum is typically deposited on monolayers of PS I by photoreduction of Pt⁴⁺ ions in solution, in the presence of an electron donor (e.g. 20 mM Na-ascorbate) and an electron carrier (e.g. 2,6 Dichloroindophenol (DCIP)) under light.

In Example 2, PSIs are connected to a solid surface so as to generate an orientated monolayer as described for Example 1. The monolayer is then platinized. Next, pre-platinized PSIs are added to the monolayer resulting in the generation of multilayers of PSI.

Methods of generating pre-platinized PSIs are known in the art—see for example [Millsaps, J. F.; Bruce, B. D.; Lee, J. W.; Greenbaum, E. Photochemistry and Photobiology 2001, 73, 630-635]. Platinum is typically deposited on a suspension of PS I by photoreduction of Pt⁴⁺ ions in solution, in the presence of an electron donor (e.g. 20 mM Na-ascorbate) and an electron carrier (e.g. 2,6 Dichloroindophenol (DCIP)) under light.

It will be appreciated that when the photocatalytic unit is bound to a metal nanoparticle, a surface may be fabricated by a multilayer arrangement of photocatalytic units by alternately layering with photocatalytic unit and metal nanoparticle. In such an arrangement, the nanoparticle also conducts current generated in the serially arranged photocatalytic units.

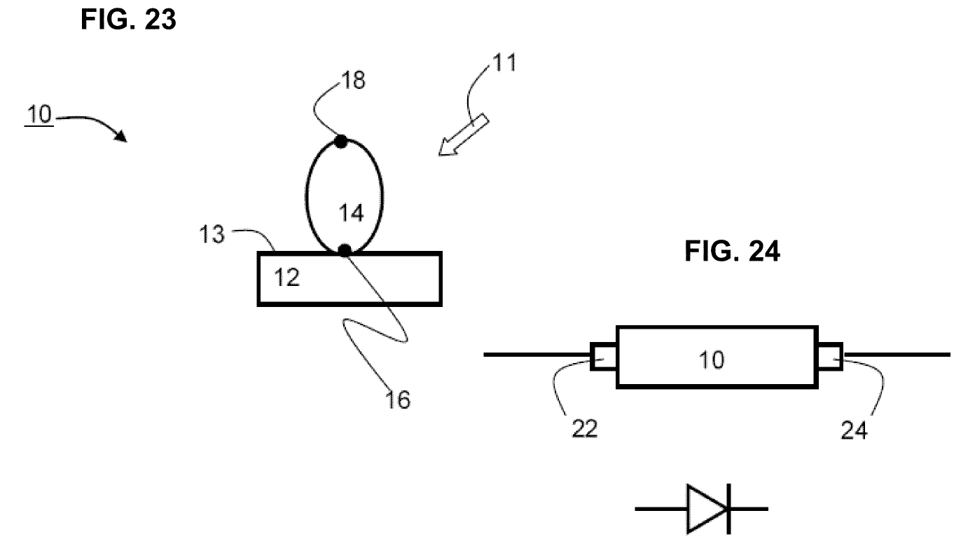

Reference is now made to FIG. 23, which is a schematic illustration of an optoelectronic device 10, according to various exemplary embodiments of the present invention. Device 10 comprises a solid support 12 and a plurality of nanostructures 14 attached to a surface 13 of support 12. At least a few of nanostructures 14 comprise one or more solid conductive nanoparticles (e.g., semiconductor nanoparticles, conductive nanoparticles, nanoshells, etc.) bound to a photocatalytic unit of a photosynthetic organism, as further detailed hereinabove.

The photocatalytic units of nanostructures 14 are preferably modified so as to facilitate covalent attachment of units 14 to surface 13, while maintaining the photocatalytic activity as further detailed hereinabove.

Being compose in part of nanostructures 14, optoelectronic device 10 facilitates light induced electron transfer. Upon excitation by light 11, an electron transfer occurs from a donor site 16, across multiple intermediate steps to an acceptor site 18, within a period of time which can be from several hundreds of picoseconds to a few microseconds, depending on the type of photocatalytic units. The frequency of light which induces the electron transfer depends on the photosynthetic organisms from which units 14 are obtained. For example, when photocatalytic units of green plants or green bacteria are employed, device 10 is sensitive to green light having wavelength of from about 400 nm to about 750 nm, when photocatalytic units of cyanobacteria are employed, device 10 is sensitive to cyan light having wavelength of from about 400 nm to about 500 nm, when photocatalytic units of red algae are employed, device 10 is sensitive to red light having wavelength of from about 650 nm to about 700 nm and when photocatalytic units of purple bacteria are employed, device 10 is sensitive to purple light having wavelength of from about 400 nm to about 850 nm.

Optoelectronic device 10 can be used in the field of micro- and sub-microelectronic circuitry and devices including, but not limited to spatial imaging devices, solar batteries, optical computing and logic gates, optoelectronic switches, diodes, photonic ND converters, and thin film “flexible” photovoltaic structures.

Reference is now made to FIG. 24, which is a schematic illustration of a photodiode device 20, according to various exemplary embodiments of the present invention. One skilled in the art will recognize that several components appearing in FIG. 23 have been omitted from FIG. 24 for clarity of presentation. Photodiode device 20 comprises optoelectric device 10, and two electrical contacts 22 and 24 being in electrical communication with donor site 16 and acceptor site 18, respectively. Electrical communication with donor site 16 can be established, for example, by connecting an electrically conductive material to support 12 or surface 13. The acceptor site can be covalently bound by formation of sulfide bond between the modified polypeptides of the photocatalytic unit of the nanostructures of the present embodiments (e.g. W31C in PsaC subunit of PS I) and the top deposited metal electrode. Platinized photocatalytic units at the acceptor side can make a metal to metal electrical connection with a top electrode deposited by evaporation of thin metal electrode. Deposition of electrically conductive polymer on top of the photocatalytic monolayer or the platinized photocatalytic monolayer can serve as a top electrode. A symbolic illustration of the photodiode is illustrated at the bottom of FIG. 24. It will be appreciated that when the nanostructure of the present embodiments comprises an electrically conductive nanoparticle, the conducing nanoparticle itself can make a metal to metal electrical connection with a top electrode.

In use, the nanostructures are irradiated by light hence being excited to efficient charge separation of high quantum efficiency, which is typically above 95%. Contacts 22 and 24 tap off the electrical current caused by the charge separation. Depending on the voltage applied between contacts 22 and 24, photodiode device 20 can be used either as a photovoltaic device, or as a reversed bias photodiode.

Specifically, in the absence of external voltage, photodiode device 20 enacts a photovoltaic device which produces current when irradiated by light. Such device can serve as a component in, e.g., a solar cell.

When reverse bias is applied between contacts 22 and 24, photodiode device 20 maintains high resistance to electric current flowing from contact 24 to contact 22 as long as photodiode device 20 is not irradiated by light which excites the nanostructures. Upon irradiation by light at the appropriate wavelength, the resistance is significantly reduced. Such device can serve as a component in, e.g., a light detector.

Optoelectronic device 10 can also serve as a solar cell, when no bias voltage is applied. Upon irradiation of the nanostructures, the charge-separated state results in internal voltage between donor site 16 and acceptor site 18. The internal voltage can be tapped off via electrical contacts at donor site 16 and acceptor site 18. If the current circuit is closed externally, the current flow is maintained through repeated light-driven charge separation in the solar cell.

The generated polarized charge-separated state of device 10 can also be utilized for in a molecular transistor. Specifically, device 10 can serve as a light-charged capacitor enacting a gate electrode which modifies the density of charge carriers in a channel connected thereto.

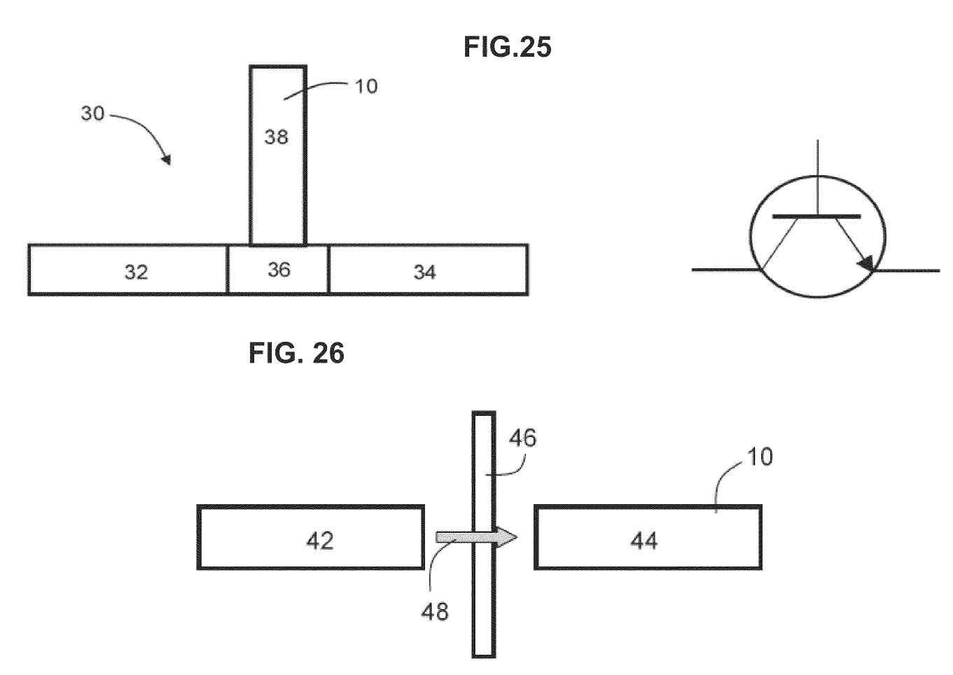

Reference is now made to FIG. 25, which is a schematic illustration of a phototransistor 30, according to various exemplary embodiments of the present invention. Phototransistor 30 comprises a source electrode 32, a drain electrode 34, a channel 36 and a light responsive gate electrode 38. Gate electrode 38 preferably comprises optoelectronic device 10. Channel 36 preferably has semiconducting properties such that the density of charge carriers can be varied.

In the absence of light, channel 36 does not contain any free charge carriers and is essentially an insulator. Upon exposure to light, the nanostructures of device 10 generate a polarized charge-separated state and the electric field caused thereby attracts electrons (or more generally, charge carriers) from source electrode 32 and drain electrode 34, so that channel 36 becomes conducting. Thus, phototransistor 30 serves as an amplifier or a switching device where the light controls the current flowing from source electrode 32 and drain electrode 34.

The electrodes can be made of any electrically conductive material, such as, but not limited to, gold. The inter-electrode spacing determines the channel length. The electrodes can be deposited on a semiconductor surface to form the source-channel-drain structure. The gate electrode can be formed from the nanostructures of the present embodiments as further detailed hereinabove. A symbolic illustration of the phototransistor is illustrated at the right hand side of FIG. 25.

As will be appreciated by one ordinarily skilled in the art, phototransistor 30 can operate while gate electrode 38 is left an open circuit because the gating is induced by photons impinging on electrode 38. Phototransistor 30 can be used as a logical element whereby the phototransistor can be switched to an on state by the incident light. In addition, phototransistor 30 can be used as the backbone of an image sensor with large patterning possible due to a strong variation of the drain current with the spatial position of the incident light beam. Several phototransistors, each operating at a different wavelength as further detailed hereinabove can be assembled to allow sensitivity of the image sensor to color images. The charge storage capability of the structure with further modifications known to one skilled in the art of conventional semiconductors can be exploited for memory related applications.

Photodiode 20 and/or phototransistor 30 can be integrated in many electronic circuitries. In particular, such devices can be used as building blocks which can be assembled on a surface structure to form a composite electronic assembly. For example, two or more photodiodes or phototransistors can be assembled on a surface structure to form a logic gate, a combination of logic gates or a microprocessor.

Reference is now made to FIG. 26 which is a simplified illustration of an optocoupler 40, according to various exemplary embodiments of the present invention. Optocoupler 40 is particularly useful for transferring signals from one element to another without establishing a direct electrical contact between the elements, e.g., due to voltage level mismatch. For example, optocoupler 40 can be used to establish contact free communication between a microprocessor operating at low voltage level and a gated switching device operating at high voltage level.

According to an embodiment of the present invention optocoupler 40 comprises an optical transmitter 42 and an optical receiver 44. Transmitter 42 can be any light source, such as, but not limited to, a light emitting diode (LED). Receiver 44 preferably comprises optoelectronic device 10, and can be, for example, a photodiode (e.g., photodiode 20) or a phototransistor (e.g., phototransistor 30). Transmitter 42 is selected such that the radiation emitted thereby is at sufficient energy to induce charge separation between donor site 16 and acceptor site 18 of device 10.

Transmitter 42 and receiver 44 are kept at optical communication but electrically decoupled. For example, transmitter 42 and receiver 44 can be separated by a transparent barrier 46 which allows the passage of light but prevents any electrical current flow thereacross. Transmitter 42 and receiver 44 preferably oppose each other such that the radiation emitted from transmitter 42 strikes receiver 44.

Triggered by an electrical signal, transmitter 42 emits light 48 which passes through barrier 46 and strikes receiver 44. In turn, receiver 44 generates an electrical signal which can be tapped off via suitable electrical contacts as further detailed hereinabove. Thus optocoupler 40 successfully transmits to its output (receiver 44) an electrical signal applied at its input (transmitter 42), devoid of any electrical contact between the input and the output.

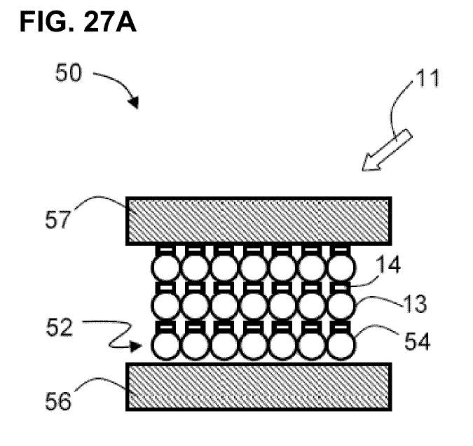

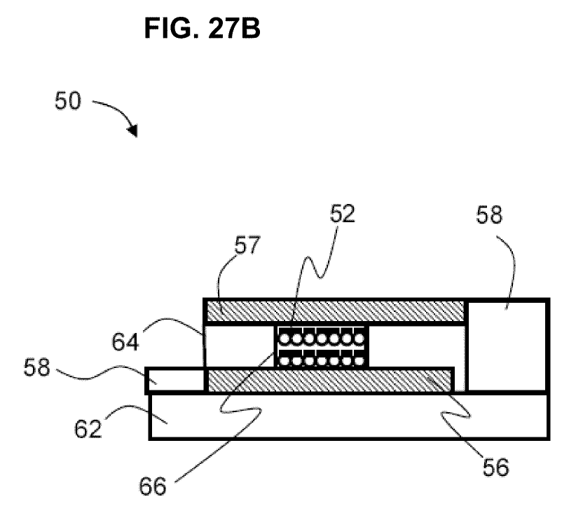

Reference is now made to FIGS. 27A-B, which are simplified illustrations of an optoelectronic device 50, according to various exemplary embodiments of the present invention.

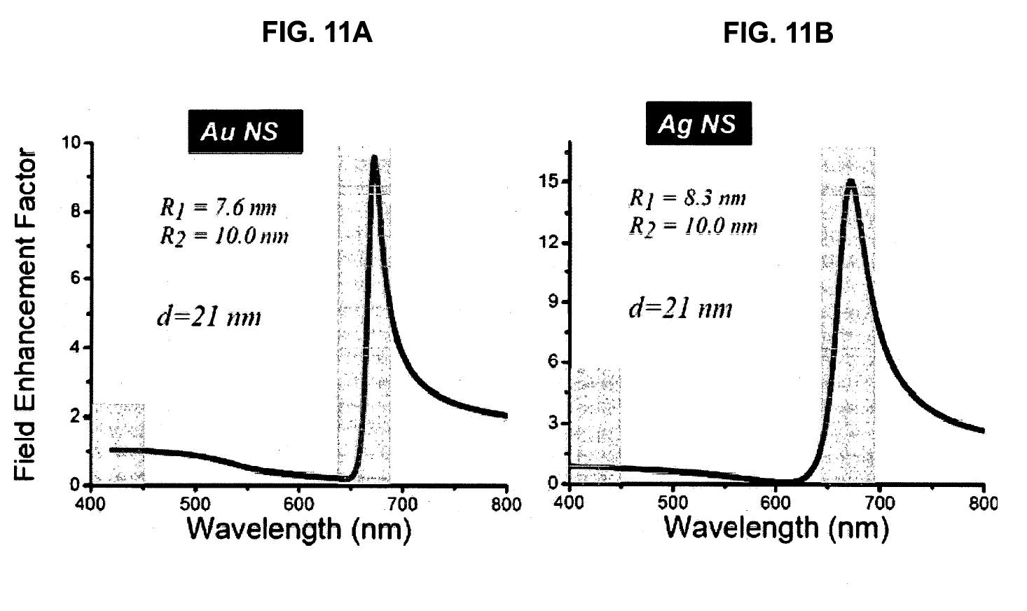

In its simplest configuration, device 50 comprises one or more layers 52 of nanostructures 54, which are optionally and preferably similar or the same as nanostructures 14 described above. Nanostructures 54 are interposed between two electrodes 56 and 57. In the representative example shown in FIG. 11, electrode 57 is light transmissive. Electrode 56 can be light transmissive, light reflective or light absorptive.

In use, electrode 57 is irradiated by light 11 which penetrates electrode 57 to impinge on layers 52. Each nanostructure absorbs the energy of the light resulting in an electric dipole directed from electrode 56 to electrode 57 or vice versa. A potential difference is thus generated between electrodes 56 and 57. Electrical current caused by the potential difference can then be tapped off by electrical contacts as further detailed hereinabove. Thus, layers 56 and 57 serve as electron and hole injection contacts and device 50 generates a photocurrent in response to light.

In various exemplary embodiments of the invention the work functions of electrodes 56 and 57 differ. Preferably, the work function of electrode 56 is lower than the work function of electrode 57. The work function of a substance is defined as the minimal energy required for removing an electron from the substance into the vacuum. According to an embodiment of the present invention, layer 56 is a low work function electrode.

As used herein, the term “low work-function” refers to a work-function of 4.5 eV or less, more preferably 4 eV or less.

Suitable low work function materials include, without limitation, alkaline metals, Group 2A, or alkaline earth metals, and Group III metals including rare earth metals and the actinide group metals. Also contemplated are the Group IB metals, metals in Groups IV, V and VI and the Group VIII transition metals. More specific examples of low work function materials, include, without limitation, lithium, magnesium, calcium, aluminum, indium, copper, silver, tin, lead, bismuth, tellurium and antimony.

According to a preferred embodiment of the present invention aluminum, layer 57 is a high work function electrode.

As used herein, the term “high work-function” refers to a work-function of 4.5 eV or more, more preferably 5 eV or more.

Suitable high work function materials include materials having any one of InSnO₂, SnO₂ and zinc oxide (ZnO) metal alloys. Other than these alloys, oxides of Sn and Zn may also be contained in the material of electrode 57.

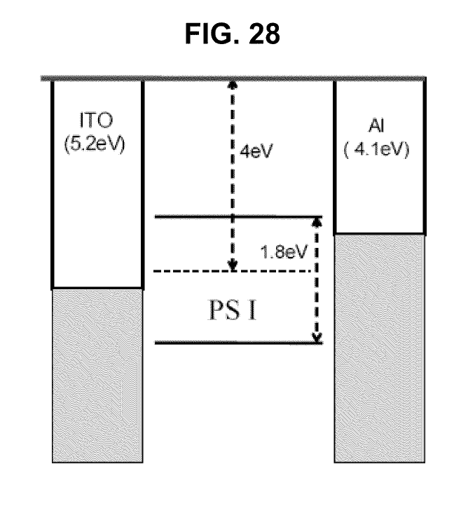

FIG. 28 illustrates an energy-level diagram in the preferred embodiment in which electrode 56 is made of aluminum and electrode 57 is made of ITO. The internal electric field generated between the electrodes is sufficiently high to generate electric field that higher than the electron-cation pair excitonic energy.

According to an embodiment of the present invention device 50 comprises a dielectric layer 64 deposited on electrode 56. Dielectric layer has a cavity 66 which exposes electrode 56. In this embodiment, layer(s) 52 are preferably placed in cavity 66 such that the nanostructures contact electrode 56 at the base of the cavity and electrode 57 at the top of the cavity. Device 50 preferably comprises a substrate 62 which serves for carrying electrode 56 and layer 64. Two or more electrical contacts 58 are preferably attached to or formed on substrate 62. Contacts 58 are in electrical communication with electrodes 56 and 57 so as to tap off the electrical current of device 50.

In various exemplary embodiments of the invention the sizes of the above electronic devices (including, without limitation, the optoelectronic device, solar cell, photodiode, phototransistor, logic gate and optocoupler) are in the sub millimeter range. Preferably, the size of the electronic devices is from about 0.1 nm to about 100 μm, more preferably, from about 0.1 nm to about 1 μm.

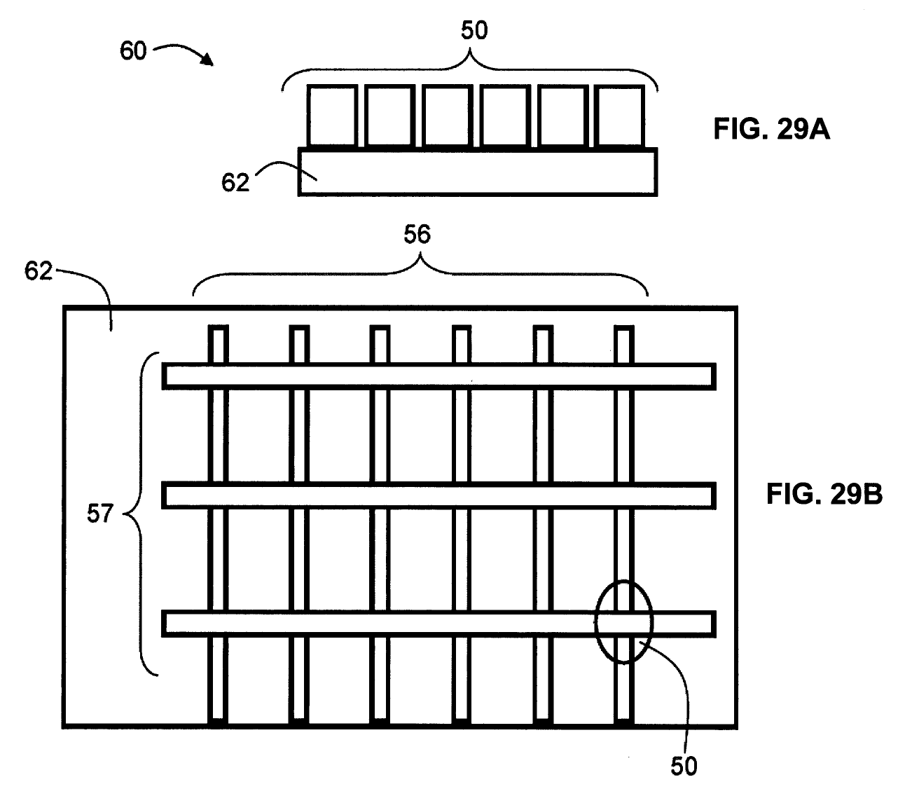

Reference is now made to FIGS. 29A-B, which are schematic illustrations of an optoelectronic array 60, according to various exemplary embodiments of the present invention. Optoelectronic array 60 comprises several optoelectronic devices similar to device 50 arranged array-wise on a substrate 62, for example, a silicon substrate or the like. The advantage of using an optoelectronic array is that such configuration can facilitates up-scaling of the physical dimensions of the optoelectronic device to amplify the photovoltaic signal. The dimensions of such optoelectronic array can be from several microns to a few centimeters.

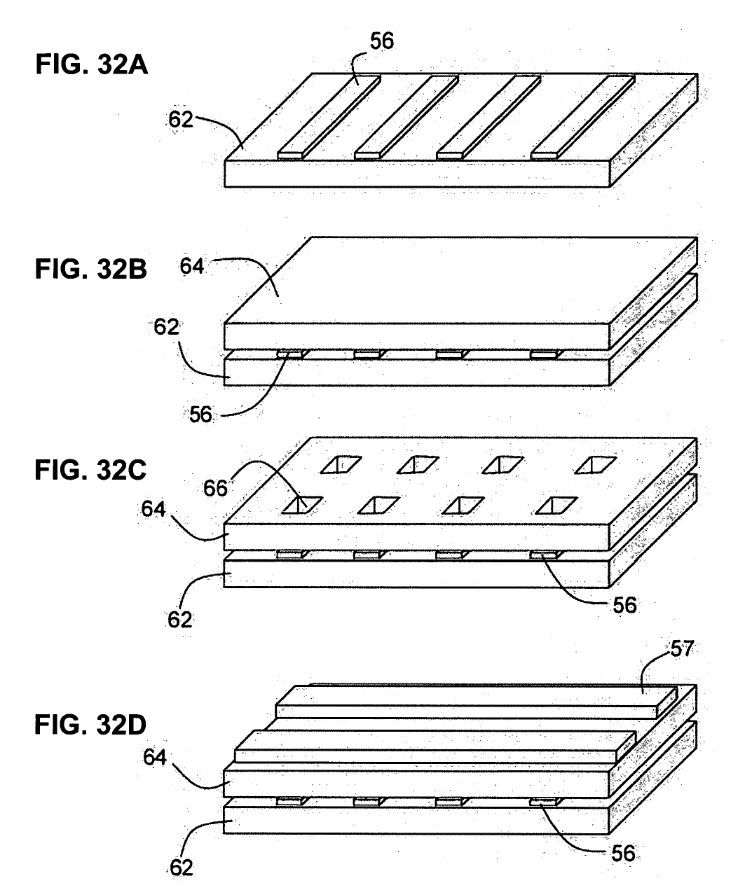

The electric configuration between the optoelectronic devices of array 60 depends on the desired output. For current output, the preferred electric configuration is serial, whereas for voltage output a parallel configuration is more preferred. The arrangement of the optoelectronic devices on substrate 62 is preferably such that several optoelectronic devices share the same electrodes. This can be achieved in any geometrical arrangement. For example, referring to FIG. 29B, two conductive layers and a dielectric layer separating one layer from the other can be deposited on substrate 62. One conductive layer can include electrodes of the type of, e.g., electrode 56, and another conductive layer can include electrodes of the type of, e.g., electrode 57. The electrodes of the conductive layers are preferably arranged in orthogonal or any other no-parallel directions. The photoactive nanostructures of device 50 are introduced into cavities formed in the dielectric layer at the intersections between the electrodes of one layer and the electrodes of the other layer, such that each such intersection defines one optoelectronic device. A preferred process for fabricating array 60 is provided hereinunder with reference to FIG. 32A-D.

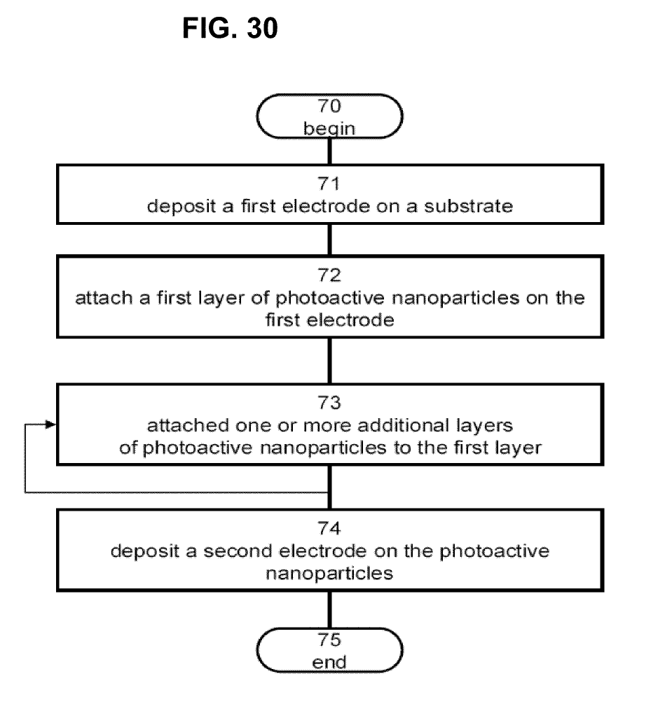

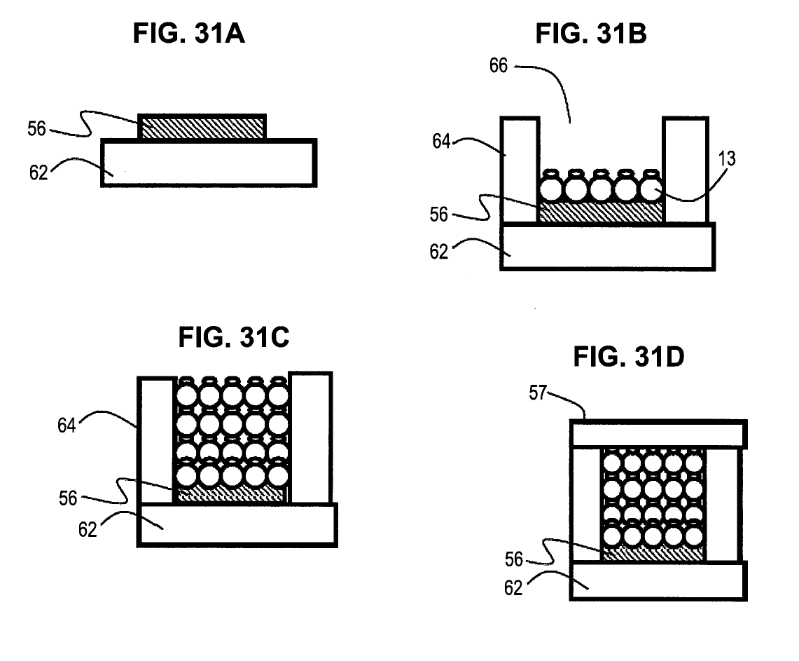

Reference is now made to FIGS. 30 and 31A-D which are a flowchart diagram (FIG. 30) and schematic process illustrations (FIGS. 31A-D) of a method suitable for fabricating an optoelectronic device, according to various exemplary embodiments of the present invention.

It is to be understood that, unless otherwise defined, the method steps described hereinbelow can be executed either contemporaneously or sequentially in many combinations or orders of execution. Additionally, one or more method steps described below are optional and may not be executed.

The method begins at step 70 and optionally and preferably continues to step 71 in which a first electrode is deposited on a substrate. FIG. 31A illustrate first electrode 56 deposited on a substrate 62. The first electrode, as stated, is preferably an electron-injection electrode which can be light transmissive, light reflective or light absorptive as desired. Step 71 can be executed by evaporation followed by photolithography and etching. For example, gold metal can be evaporated on a substrate silicon dioxide layer. The gold layer can then patterned by photolithography according to the desired shape of the first electrode. Subsequently, the electrode can be shaped by etching.

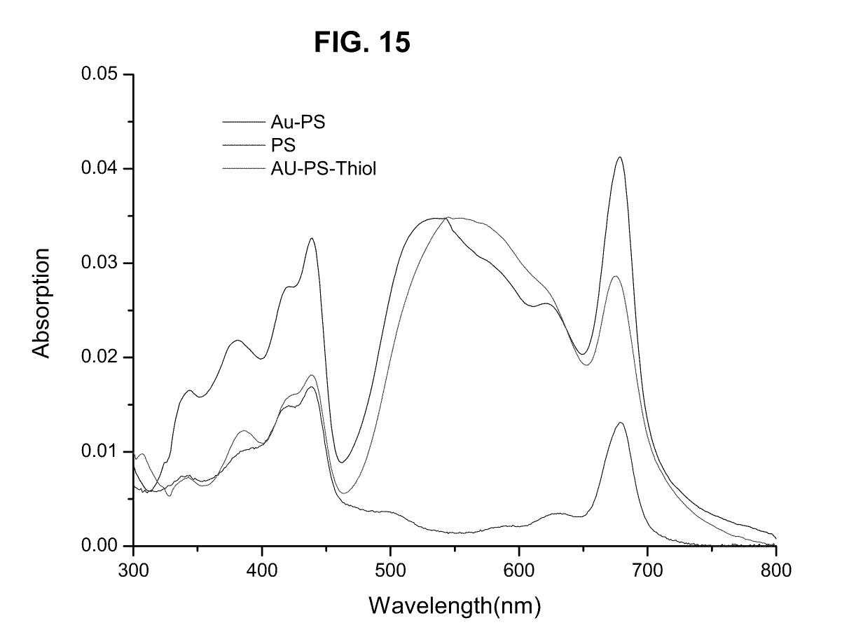

The method continues to step 72 in which nanostructures are covalently attached to the first electrode to provide a first layer of photoactive nanostructures as further detailed hereinabove. In some embodiments of the present invention the top side of the nanostructures preferably comprises a conducting moiety to allow attachment of other nanostructures. The method then continues to step 73 in which one or more layers of nanostructures are attached to the first layer electrode (see FIG. 15b), to provide a plurality of layers of nanostructures. Step 73 can be repeated one or more time, depending on the number of nanostructure layers of the device.

Step 72 preferably comprises fabrication of a cavity 66, e.g., by forming a cavity through a dielectric layer 64 on top of first electrode 56 and substrate 62. The dielectric layer can be made of any dielectric material suitable for the process by which the cavity is formed. For example, a layer of silicon nitride can be deposited on top of the first electrode, e.g., using Chemical Vapor Deposition (CVD), physical vapor deposition (PVD), or atomic layer deposition (ALD). The cavity can then be formed in the dielectric layer (silicon nitride, in the present example) by photolithography followed by etching. In any event, cavity 66 is formed such that first electrode 56 is exposed on the base of the cavity, to allow adsorption of the nanostructures on the first electrode.

The preferred adsorption technique depends on the type of nanostructures. In various exemplary embodiments of the invention light induced adsorption is employed. When the nanostructures comprise photocatalytic units having a modified polypeptide, the nanostructures attach to the first electrode via the amino acids at the modified site. For example, thiolated PS I units can be attached via their thiol moiety to form a stable oriented self assembled monolayer (SAM). Light induced adsorption can be used to adsorb the PS I nanostructures into a dense layer.

Chemical bonding to the second electrode of the device can be improved by photoreducing Pt⁴⁺ ions in solution by PS I monolayer. Such a procedure was earlier used for platinization of PS I in suspension [Millsaps, J. F.; Bruce, B. D.; Lee, J. W.; Greenbaum, E. Photochemistry and Photobiology 2001, 73, 630-635]. The procedure results in local deposition of Pt at the electron donor end of the protein. A fresh incubation of the platinized monolayer with cysteine mutants of PS I results in formation of sulfide bond between the cysteine in the PS I and the platinized top of the monolayer to form a second oriented SAM. These cycles are preferably repeated so as to form of an oriented multilayer inside the cavity (see FIG. 31C).

The method continues to step 74 in which the second electrode is deposited on the layer(s) of photoactive nanoparticles (see FIG. 31D). The second electrode, as stated, is preferably a hole-injection light transmissive electrode and it can be any electrode as long as it is capable of functioning as an anode so as to inject holes into the layers of nanoparticles. Preferably, the second electrode comprises ITO which can be deposited by sputtering, electron beam vapor deposition, ion plating, indirect evaporation process etc. In various exemplary embodiments of the invention ITO clusters are deposited on the nanoparticles with relatively very low momentum and temperature, so as to prevent or minimize the destruction of the nanoparticles.

The method ends at step 75.

Reference is now made to FIGS. 32A-D which are schematic illustrations of a preferred process for an optoelectronic array, according to various exemplary embodiments of the present invention. With reference to FIG. 32A, a plurality of electrodes of the type of, e.g., electrode 56, is deposited on substrate 62. The technique for depositing the electrodes can be similar to the technique described above. For example, a conductive layer can be evaporated on the substrate and, photolithography followed by etching can be employed to form the electrodes on the evaporated layer. In the simplified illustration shown in FIG. 32A, electrodes 56 are conveniently shaped as a plurality of parallel stripes, but it is not intended to exclude any other shape for the electrodes.

With reference to FIGS. 32B-C, dielectric layer 64 is deposited on top of electrodes 56 and a plurality of cavities 66 are formed in dielectric layer 64 by photolithography followed by etching to expose electrode 56 as further detailed hereinabove.

Once the cavities are formed, the nanostructures can be introduced into the cavities as further detailed hereinabove. A plurality of electrodes of the type of, e.g., electrode 57, is then deposited on layer 64 so as to contact the nanostructures in cavities 66. Electrodes 57 are illustrated in FIG. 32D as a plurality of parallel stripes, substantially orthogonal to electrodes 56. Other shapes for electrodes 57 are also contemplated, provided the nanostructures in the cavities interconnect electrodes 56 and 57.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and the include plural references unless the context clearly dictates otherwise. For example, the term “a nanoparticles” or “at least one nanoparticle” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find calculated and experimental support in the following examples.

### EXAMPLES

Reference is now made to the following examples, which together with the above descriptions illustrate some embodiments of the invention in a non limiting fashion. Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Fabrication of Oriented Multilayers of Photosystem I Proteins on Solid Surfaces by Auto-Metallization

Materials and Methods

Site-directed mutagenesis: For site-directed mutagenesis in the psaB gene from Synechocystis sp. PCC 6803 was induced by homologous recombination using plasmids pZBL for induction of cysteine Y634C mutations and pBLΔB for psaB interruption in recipient cells, as previously described [L. Frolov, Y. Rosenwaks, C. Carmeli, I. Carmeli, Adv. Mater. 2005, 17, 2434; M. T. Zeng, X. M. Gong, M. C. Evans, N. Nelson, C. Carmeli, Biochim. Biophys. Acta 2002, 1556, 254; X. M. Gong, R. Agalarov, K. Brettel, C. Carmeli, J. Biol. Chem. 2003, 278, 19141].

Isolation and characterization of PS I complexes: PS I was isolated from thylakoid membranes by solubiliztion with n-dodecyl β-D-maltoside and purification on DEAE-cellulose columns and on a sucrose gradient. The isolation of PS I, the analysis chlorophyll content and photochemical activity determined by flash-induced transient oxidation of P700 at ΔA820 and at ΔA700 nm were as described [X. M. Gong, R. Agalarov, K. Brettel, C. Carmeli, J. Biol. Chem. 2003, 278, 19141]. In both the cysteine mutant and the native PS I, a half-time of 25 ms for the reduction of P700 was recorded. Surface-exposed cysteines on PS I were probed by biotin-maleimide, as previously described [J. Sun, A. Ke, P. Jin, V. P. Chitnis, P. R. Chitnis, in Meth. Enzy., Academic Press 1998, 124]. Biotin-labeled PS I complexes were dissociated and separated by sodium dodecyl sulfate polyacrylamide gel electrophoresis. For immunoblot detection, protein samples were transferred from the gel to nitrocellulose and reacted with peroxidase-conjugated avidin, then developed with enhanced chemiluminescence reagents.

Atomic Force Microscopy (AFM)-All measurements were carried out with a commercial AFM (Nanoscope® IIIa MultyMode™ with Extender™ Electronics Module, Veeco Instruments). The topography measurements were conducted in a tapping mode at a cantilever resonance frequency of 300 kHz.

Platinization of PS I: Pt was deposited on monolayers of PS I by photoreduction of Pe⁴⁺ ions in solution. Slides of PS I monolayer on gold were incubated in a reaction medium containing: 0.2 mM PtCl₆²⁻, 50 mM KH₂PO₄, pH 8, 20 mM Na-ascorbate as an electron donor and 50 μM 2,6 Dichloroindophenol (DCIP) as an electron carrier. The reaction was illuminated by a tungsten lamp, with intensity of 35 Watt per cm², for various times intervals between 5 to 30 min at 20° C. Slides were then washed with distilled water and dried with ultrapure nitrogen. For multi-layer formation, slides were sequentially incubated in solutions containing PS I, washed and then platinized.

Kelvin Probe Force Microscopy (KPFM): The KPFM set-up is based on a commercial AFM model NTMDT, equipped with a custom-made 1300-nm wavelength feedback laser to prevent any sample-induced photovoltage. Most CPD measurements were conducted in a nitrogen glove box. A comparison with an in-situ peeled pyrolitic graphite standard (OPG) enabled the extraction of the actual work function of all measured samples. The electrostatic force is measured in the so-called ‘lift mode’; in this mode, after the topography is measured, the tip is retracted from the sample surface to a fixed height. The oscillation of the tip induced by the piezo is stopped and an AC bias is applied to the cantilever at the same frequency previously used for the topography measurements in the tapping mode. The CPD is extracted in the conventional way by nullifying the output signal of a lock-in amplifier, which measures the electrostatic force at the first resonance frequency [O. Vatel, M. Tanimoto, J. Appl. Phys. 1995, 77, 2358]. AFM topography and the corresponding KPFM electric potential were recorded in sequential scans at a scan rate of 1 Hz; 512 lines were scanned in two segments over the sample area to form a two-dimensional image. A diode laser (λ=670 nm, 40 mW) was switched on and off at the indicated segment of the scan.

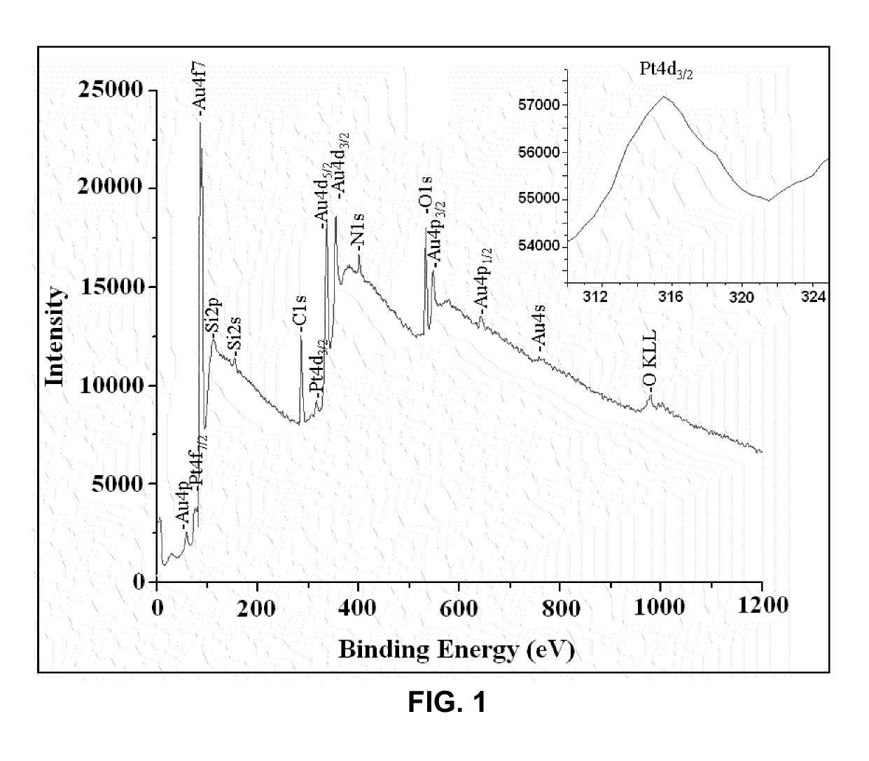

X-ray photoelectron spectroscopy (XPS)-XPS utilizes photo-ionization and energy-dispersive analysis of the emitted photoelectrons to study the composition and electronic state of the surface region of a sample. In XPS, the photon is absorbed by an atom in a molecule or solid, leading to ionization and the emission of a core (inner shell) electron. This experiment was performed in order to determine the element composition of the surface. As expected, Pt appeared only in PS I monolayer slides that were reacted with [PtCl₆]²⁻, and protein nitrogen was detected on slides containing PS I monolayer. The presence of Si is due to contamination from the silicon wafer. The composition of elements in the platinized PS I monolayer is portrayed in FIG. 1 and Table 1 herein below.

TABLE 1

| Element/ | | | |
|---|---|---|---|
| sample | Au | PS I (% mol) | PS I-Pt |
| C | 46.80 ± 0.94 | 47.42 ± 0.95 | 49.46 ± 0.99 |
| O | 21.09 ± 1.10 | 21.11 ± 1.10 | 22.16 ± 1.20 |
| Au | 27.40 ± 1.00 | 21.91 ± 1.20 | 17.39 ± 0.35 |
| Cu | 1.15 ± 0.56 | 0.27 ± 1.62 | 0.06 ± 0.04 |
| Si | 2.24 ± 1.20 | 3.18 ± 0.90 | 4.22 ± 0.85 |
| S | 0.83 ± 0.41 | 0.99 ± 0.38 | 0.97 ± 0.38 |
| N | 0.48 ± 0.48 | 5.14 ± 1.10 | 5.87 ± 1.06 |
| Pt | 0.00 | 0.00 | 0.90 ± 0.39 |

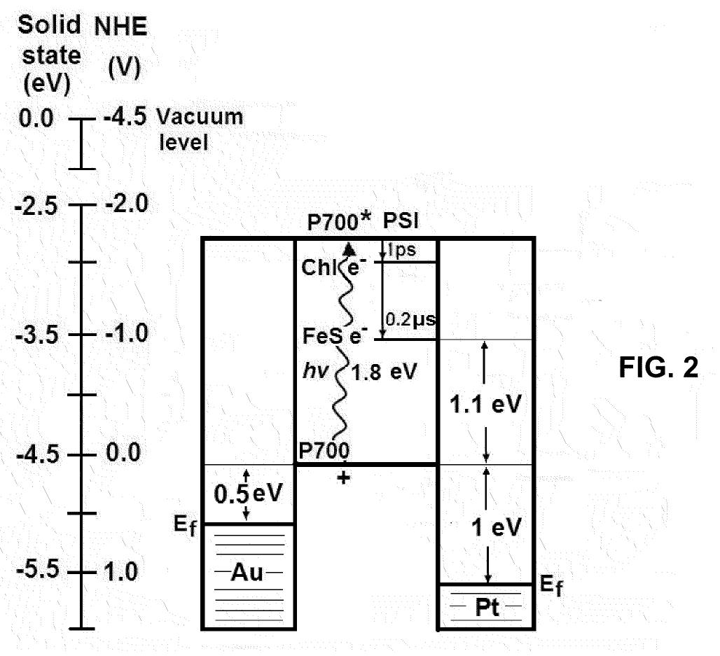

FIG. 2 is a schematic presentation of energy levels in a PS I in junction with gold and platinum. The energies were determined by measurements of CPD compared to a graphite standard and from the published work function energy. The redox levels of electron carriers in PS I were assigned according to the potential measured against normal hydrogen electrode (NHE). The redox potentials at pH 7[K. Brettel, W. Leibl, Biochim. et Biophys. Acta-Bioenerg. 2001, 1507, 100] were converted to NHE values by addition of 0.41 V. The scale on the left shows the solid state energy levels in relation to the NHE redox levels [A. J. Nozik, Annu. Rev. Phys. Chem. 1978, 29, 189]. The solid state energy levels were −5.1 and −5.6 eV [15] for gold and platinum Fermi-level (E_{f}), respectively. The energy levels in PS I were: −4.58, −2.78, −3.06, −3.52 eV for the primary electron donor (P700), excited P700*, the primary (Chl) and the final (FeS) electron acceptors, respectively.

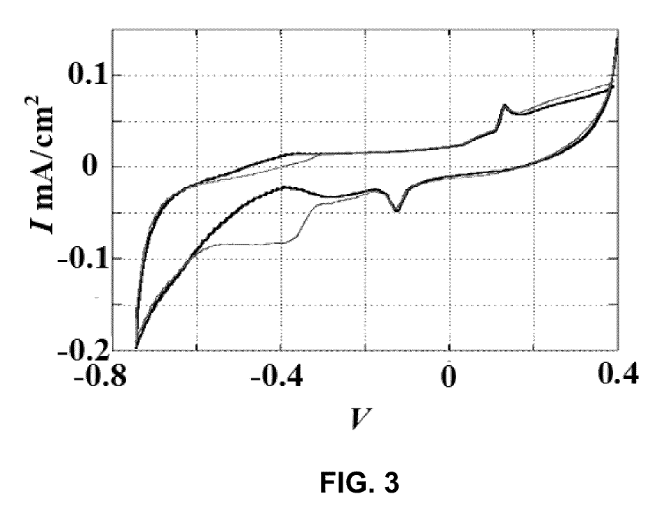

Electrochemical measurements: Electrochemistry assay was performed in order to determine the electronic coupling between the gold electrode and the PS I monolayer and multilayer slide in solution. The measuring set-up included an Ag/AgCl/1M KCl reference electrode, a Pt counter electrode and a working electrode made from a PS I monolayer on gold surface. Cyclic voltametry was used to determine the redox reactions of the surface. The electric current was measured in response to cyclic voltage changes between (+)0.4V and (−) 0.75V. In preliminary experiments a large light induced current was observed at −0.4 V (FIG. 3), a value which is slightly smaller than the redox potential of the final electron acceptor FeS. The large photocurrent measured for the PS I monolayer is indicative of a good electronic coupling between the gold electrode and PS I in solution. It is possible these results are indicative for the presence of similar electronic coupling between the gold and the dry PS I layers.

Results

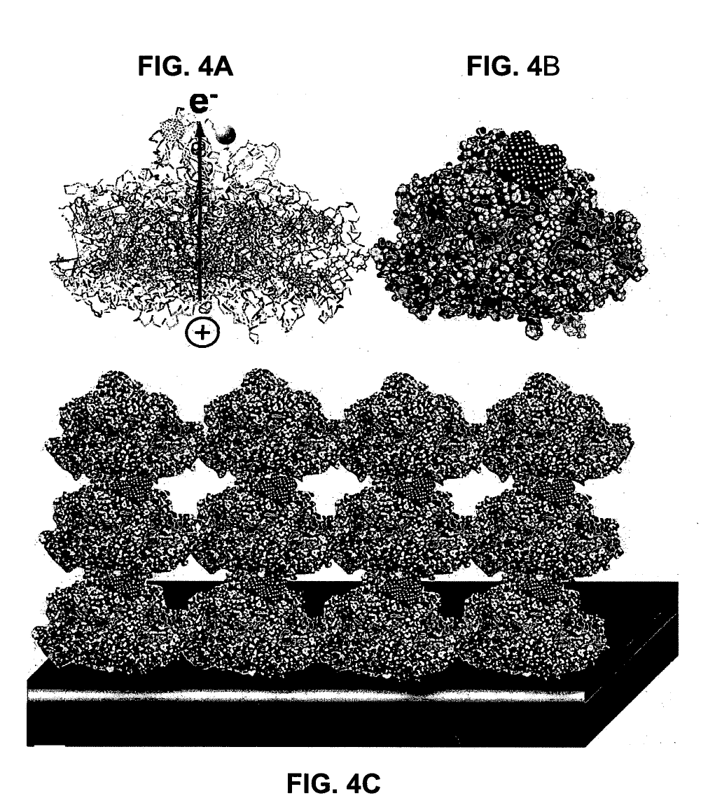

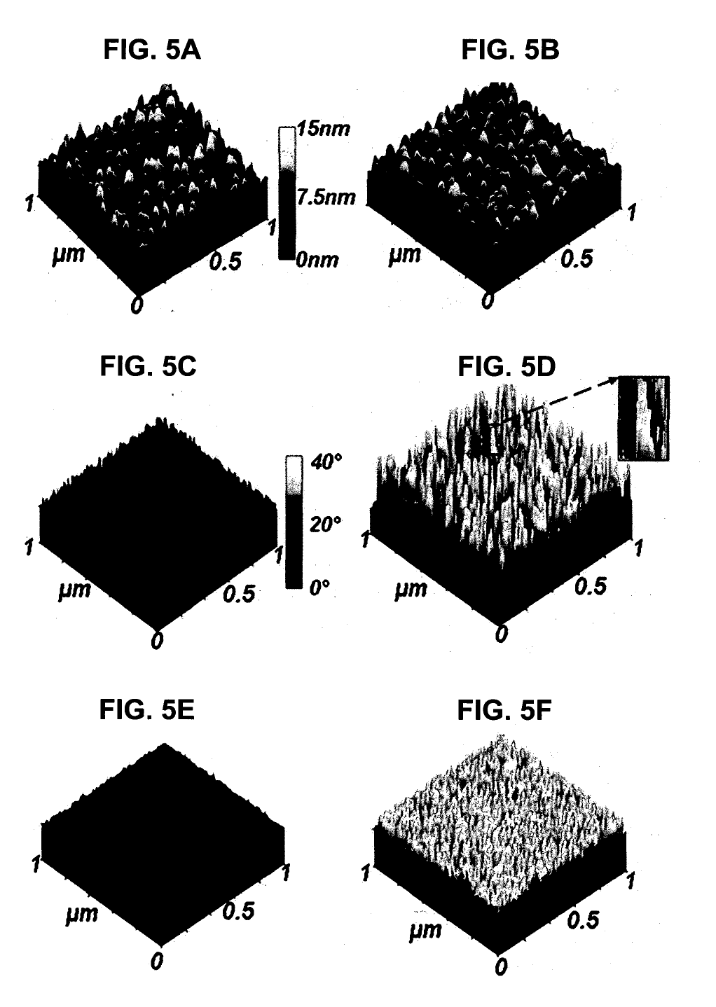

Initially, an oriented monolayer was fabricated using cysteine mutant Y634C in subunit PsaB of PS I from the cyanobacteria Synechosystis sp. PCC 6803[L. Frolov, Y. Rosenwaks, C. Carmeli, I. Carmeli, Adv. Mater. 2005, 17, 2434; M. T. Zeng, X. M. Gong, M. C. Evans, N. Nelson, C. Carmeli, Biochim. Biophys. Acta 2002, 1556, 254]. The mutated amino acid is located near P700 in the external membrane loops and does not have stereo hindrance when placed on a solid surface, assuring the formation of sulfide bonds and close electronic junction (FIG. 4A). The photochemical properties of the isolated unique PS I mutant Y634C were similar to that of the native complex. The fabrication of oriented monolayers was carried out by directly reacting the cysteine in the mutant PS I with a 150 nm thick gold surface on a silicon slide to form an Au-sulfide bond. Excess protein was washed and the monolayer was dried under nitrogen. AFM images clearly show a dense monolayer of 15-21 nm particles (FIG. 5A) as expected from the size of PS I as obtained by crystallography. Platinum was deposited on the protein by photo-reducing Pt⁴⁺ ions in solution by the vectorially oriented PS I layers, according to the reaction: [PtCl₆]²⁻+4e+hv=Pt↓+6Cl⁻. The source of electrons for reduction of [PtCl₆]²⁻ were the electrons from the light-activated PS I, that was continuously re-reduced by ascorbate dichlorophynol indophenol in the solution. Ascorbate did not reduce or photo-reduce Pt⁴⁺ ions in solution in the absence of PS I monolayer. AFM images of the monolayer show that the size of the PS I slightly increased because of platinization. The phase image, however, clearly demonstrated the presence of metal deposited on top of each PS I. The phase angle of PS I (FIG. 5C) increased on top of the particles following deposition of platinum (FIG. 5D); while being lower at the bottom of each platinzed PS I as a result of the lower stiffness of the protein. It is possible that the flat tops of the images of the platinized PS I were due to the formation of crystalline-like platinum patches on the top of PS I (FIG. 5D, zoom). A simulation of deposited platinum crystals of about 2 nm at the reducing end of PS I and of the assembled multilayer are shown (FIGS. 4B-C).

The first row of oriented monolayer of PS I is shown to be attached to the solid gold surface by formation of a sulfide bond between the unique cysteine at the oxidizing end of PS I. The photo-reduction of Pt⁴⁺ ions which resulted in the deposition of Pt patches at the reducing end of each PS I molecule, is used to attach the next monolayer of PS I through the formation of sulfide bonds. Digestion of the protein in the monolayer with proteinase K in solution after Pt deposition resulted in a decrease in the size of the particles in the monolayers, as would be expected. However, particles with high phase angle remained attached to the gold surface following the digestion of the protein and intensive washing with water of the platinized PS I monolayer (FIG. 5F). This procedure can be utilized for modification of metal electrode surfaces by a monolayer of platinum nano particles.

X-ray photoelectron spectroscopy (XPS) analysis of monolayers indicated, in the present work, the deposition of 427 Pt atoms per PS I in the platinized monolayer. The calculation is based on the finding of a ratio 0.9/5.87 Pt/N assuming 2786 N atoms per PS I. In order to estimate the size of the patch, it was calculated that a crystal of ˜2 nm can be formed with this number of Pt atoms (FIG. 4B) [J. R. Anderson, Structure of Metallic Catalysts, Academic Press, New York 1975]. No Pt atoms were detected in unplatinized PS I monolayers. The results of the analysis concur with the imaging of Pt patches on the PS I in the platinized monolayers.

The platinized monolayer was washed and incubated again in a solution of cysteine mutants of PS I for binding of a second layer by a formation of sulfide bond between the oxidizing end of the proteins and platinum patches on top of the PS I complexes (the reducing side). This process was repeated several times. The formation of new layers of PS I and their platinization were monitored by observation of changes in the phase angles. The electric properties of the surface of PS I monolayer were expected to be modified following deposition of metal on the surface. The present inventors therefore measured the contact potential difference (CPD) of the metalized PS I monolayer by Kelvin probe force microscopy (KPFM). Current cannot be measured by this method, because the AFM probe is raised −20 nm above the sample surface for CPD determination. The CPD and the photovoltage were determined by a novel KPFM system that uses a 1300 nm wavelength feedback laser not absorbed by PS I[O. Vatel, M. Tanimoto, J. Appl. Phys. 1995, 77, 2358]. Self-assembly of PS I monolayer caused a decrease of ˜0.7 V in the CPD of the gold surface. Such a change is a result of formation of a Schottky junction between the gold and PS I on binding of the photosystem to the metal surface. Oxidation of P700 by the metal, which has a 0.52 eV higher work function, resulted in the positively-charged PS I expressed as a decrease in the CPD (Table 2, herein below). However, deposition of Pt caused a large increase of 0.231 Vin the CPD. The increase in CPD of the platinized surface is due to a Pt work function of −5.6 eV, which affected surface potential.

TABLE 2

| | [a] CPD (dark) | CPD (light) | Photovoltage |
|---|---|---|---|
| Sample layers | (Volt) | (Volt) | (Volt) |
| Au | 0.510 ± 1.4^{e-3} | 0.527 ± 1.5^{e-3} | 0.017 ± 2.0^{e-4} |
| Au-PSI | −0.191 ± 5.2^{e-4} | 0.061 ± 3.1^{e-4} | 0.252 ± 9.0^{e-4} |
| Au-PSI-Pt | 0.040 ± 2.5^{e-4} | 0.105 ± 4.5^{e-4} | 0.065 ± 3.0^{e-4} |
| Au-PSI-Pt-PSI | −0.177 ± 5.1^{e-4} | 0.153 ± 5.0^{e-4} | 0.330 ± 1.3^{e-3} |
| Au-PSI-Pt-PSI-Pt | 0.043 ± 2.5^{e-4} | 0.123 ± 4.7^{e-4} | 0.079 ± 3.1^{e-4} |
| Au-PSI-Pt-PSI-Pt-PSI | −0.155 ± 5.1^{e-4} | 0.231 ± 8.5^{e-4} | 0.386 ± 1.4^{e-3} |

[a] The CPD of PS I and platinized PS I reaction center mono- and multi-layers surfaces were measured by KPFM [R. Shikler, N. Fried, T. Meoded, Y. Rosenwaks, Physical Review B 2000, 61, 11041]. The structure and the composition of the mono- and multi-layers and the surfaces of gold, PS I and platinized PS I are indicated as Au, -PS I and -Pt, respectively. The measurements were carried out either in dark or in light. Photovoltage was determined from the difference between the CPD in the dark and the light. The illumination was provided by a diode laser with output power of 40 mW at 670 nm. Each value is an average of 6 samples of 512 × 512 line scans of the various surfaces. All the CPD measurements were calibrated against highly-oriented, freshly-cleaved pyrolytic graphite that gave a CPD of 0.04 V.

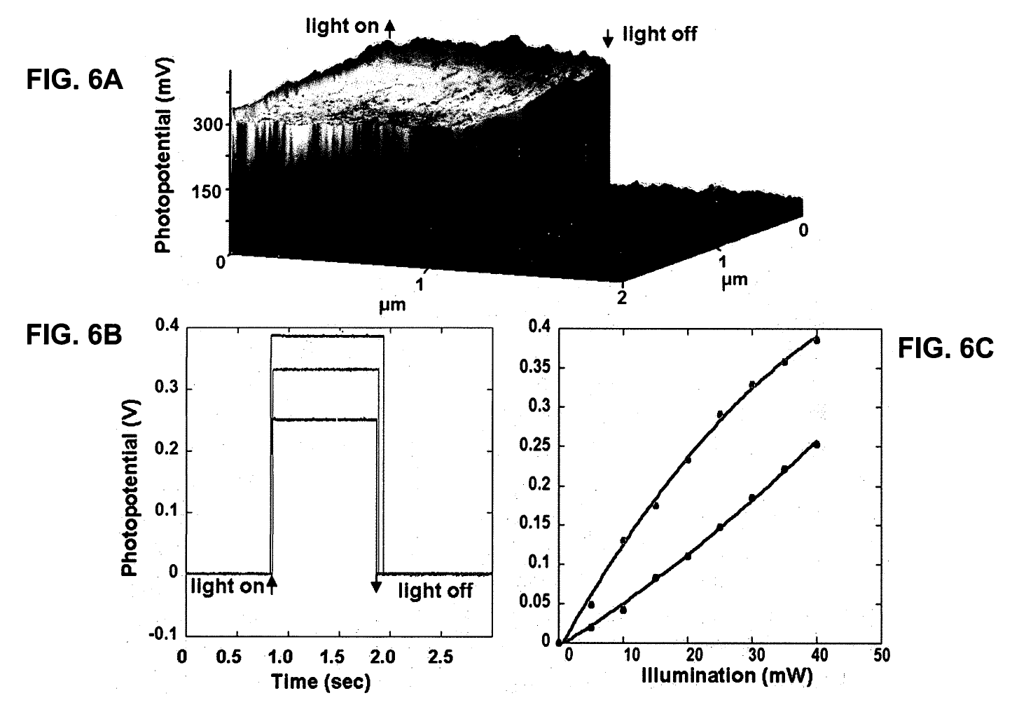

The observed increase in CPD is a clear indication of the deposition of Pt on top of the PS I and is in agreement with the observed increase in the phase angle of platinized PS I. Sequential assembly of PS I monolayer and platinization decreased and increased the CPD at approximately similar magnitudes, as was observed in the first monolayer (Table 2). These sequential changes of about 0.225 V in the CPD are independent indicative of the formation of the multilayers Illumination of PS I monolayer caused an increase of 0.252 V in the CPD due to a light-induced charge separation that drives electron transfer across the reaction center, and resulted in the appearance of a negative charge at the reducing end of the protein away from the gold surface. This value is smaller by ˜0.7 V than the expected 1.0 V difference in the energies of the primary donor P700 and the final acceptor FeS. The difference can be partially explained by a loss caused by a Schottky barrier of 0.5 eV formed between the gold and P700. The energy levels were calculated by conversion of the redox potentials at pH 7 [K. Brettel, W. Leibl, Biochim. et Biophys. Acta-Bioenerg. 2001, 1507, 100] to NHE values by addition of 0.41 V. The solid state energy levels were related to the NHE redox levels [A. J. Nozik, Annu. Rev. Phys. Chem. 1978, 29, 189]. The solid state energy levels were −5.1 and −5.6 eV [J. M. Beebe, V. B. Engelkes, L. L. Miller, C. D. Frisbie, J. Am. Chem. Soc. 2002, 124, 11268] for gold and platinum Fermi-level, respectively. The energy levels in PS I were: −4.58, −2.78, −3.06, −3.52 eV for the primary electron donor (P700), excited P700*, the primary and the final electron acceptors (FeS cluster), respectively. The photovoltage of the platinized monolayer was only 0.065 V, due to charge screening [N. D. Lang, W. Kohn, Physical Review B 1973, 7, 3541] by the coating platinum layer. A Schottky barrier of 1 eV between Pt and P700 connecting the second and the third layer caused an increase in the photopotential that was smaller than the expected additive photopotential in a serial arrangement. The decay of the photopotential was faster than the shutter-off time (0.7 ms) in all the layers (FIG. 6B), and within the decay time of light induced charge separation in PS I in solution.

Limited access of the incident light to the surface of the sample in the KPFM instrument prevented the generation of maximal photopotential. Indeed, an increase in the photovoltage as a function of light intensity did not reach saturation in the mono- and multi-layers (FIG. 6C). However, there was an increase of ˜2 fold in photopotential of the multilayers, due to both an increase in the absorption cross-section and to the electronic coupling between the serially-arranged PS I complexes. Electronic coupling between the gold electrode and the multilayers is also indicated by the light-induced photocurrent of 0.12 mA/cm² as measured by cyclic voltametry. The almost molecular recognition used for fabrication of multilayers in this work, seems to be more efficient than the approach used for the generation of enhanced photovoltage produced by the stacking of hundreds of layers of loosely-oriented bacteriorhodopsin membrane patches [G. Varo, L. Keszthelyi, Biophys. J. 1983, 43, 47; J. Shin, P. Bhattacharya, J. Xu, G. Varo, Opt. Lett. 2004, 29, 2264]. Each of the bacteriorhodopsin proteins contains only a single chromophore, and a monolayer generates ˜40 mV when excited by photons. In PS I, 120 pigment molecules harvest photon energy that is transferred in fentoseconds to a common reaction center, where a photovoltage of 1 V is generated with quantum efficiency of 1. The superior photo-electronic properties can yield an almost total absorption of visible light by the multilayers and a generation of photovoltage to be utilized in the fabrication of hybrid devices.

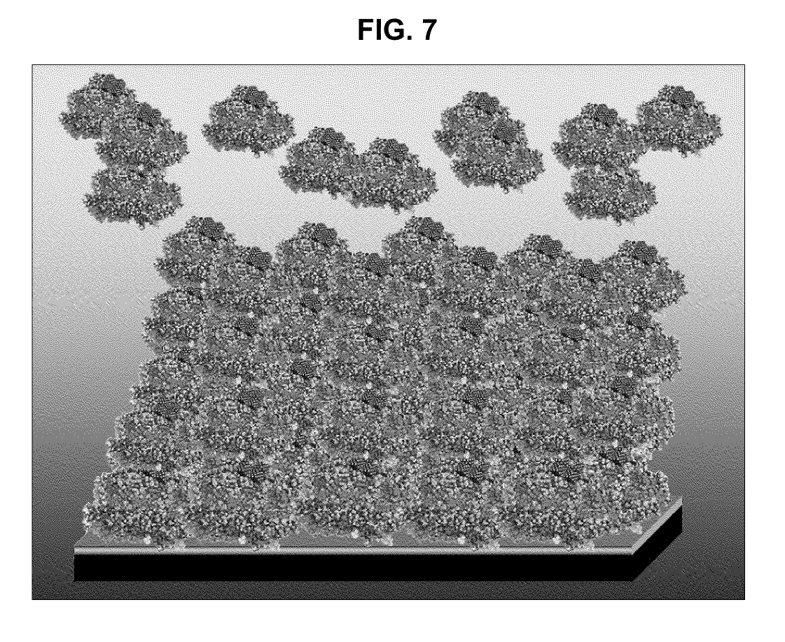

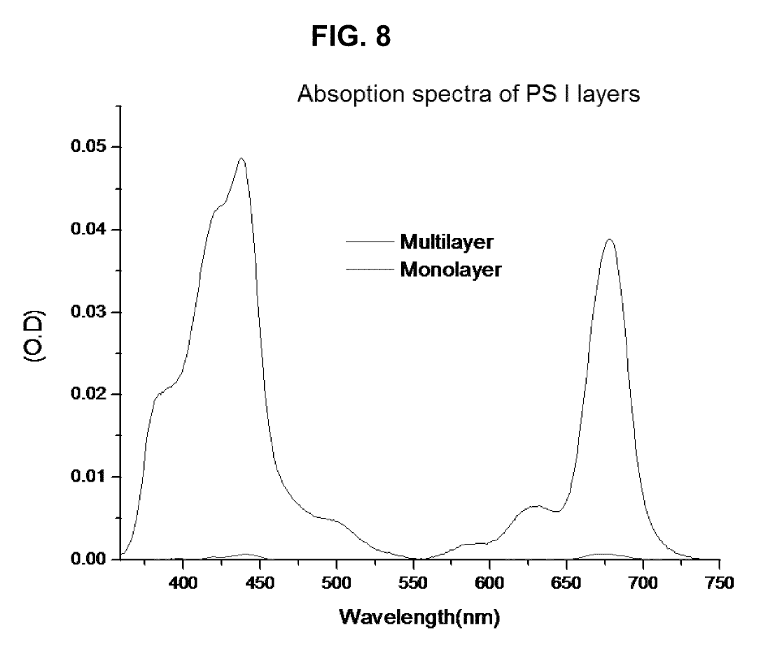

Fabrication of serially-oriented multilayers of photosynthetic reaction center photosystem I (PS I) by self assembly: Fabrication of serially-oriented multilayers of photosynthetic reaction center photosystem I (PS I) is mediated by the photo-catalytic specificity that reduced metal ions to nanometric metal patches on the reducing side of PS I forming junctions with the oxidizing end of the proteins through metal-sulfide bond of genetically-engineered cysteine mutants. The dry multilayers is utilized in hybrid bio-solid-state electronic devices in which an increase in photo-voltage, resulting from the larger absorption cross-section and the serial-arrangement of PS I. The template for the multilayers is formed by self assembly of a monolayer of cysteine mutants of PSI on metal surface which is autoplatinized by reduction of Pt⁴⁺ ions in the light in the presence of the electron donor indophynol and ascorbate forming a 2 nm platinum junction. The template is further incubated with a pre-platinized suspension of cysteine mutants of PSI to self assemble a serially oriented multilayer. Each layer is connected to the next through the formation of a sufide bond between the platinum junction of the bottom layer and the cysteine thiol of the top layer (FIG. 7). An example of the fabrication of 25 layers by self assembly is illustrated in FIG. 8. The formation of the multilayer was evaluated by measurement of the absorption spectrum. Indeed the absorption spectrum of multilayer (FIG. 8, black) indicates a formation of 25 layers is compared to the absorption spectrum of a monolayer (red). Longer incubation time results in the fabrication of a larger number of layers.

### Example 2

### A Hybrid Nanostructure Composed of a Photo-Synthetic System and Metal Nanoparticles: Plasmon Enhancement Effect

Materials and Methods

Fabrication of NP/PSI hybrids: Hybrid metal NP PS I were fabricated by direct covalent binding between NP and PSI. Cysteine mutants at the oxidizing end of PS I were bound to the metal NP by formation of sulfide bond between the cysteine thiols and the metal. NPs were fabricated by reduction of 1 mM solution of AuCl₃ and AgNO₃ by BH₄ in the presence of 5 nM PSI.

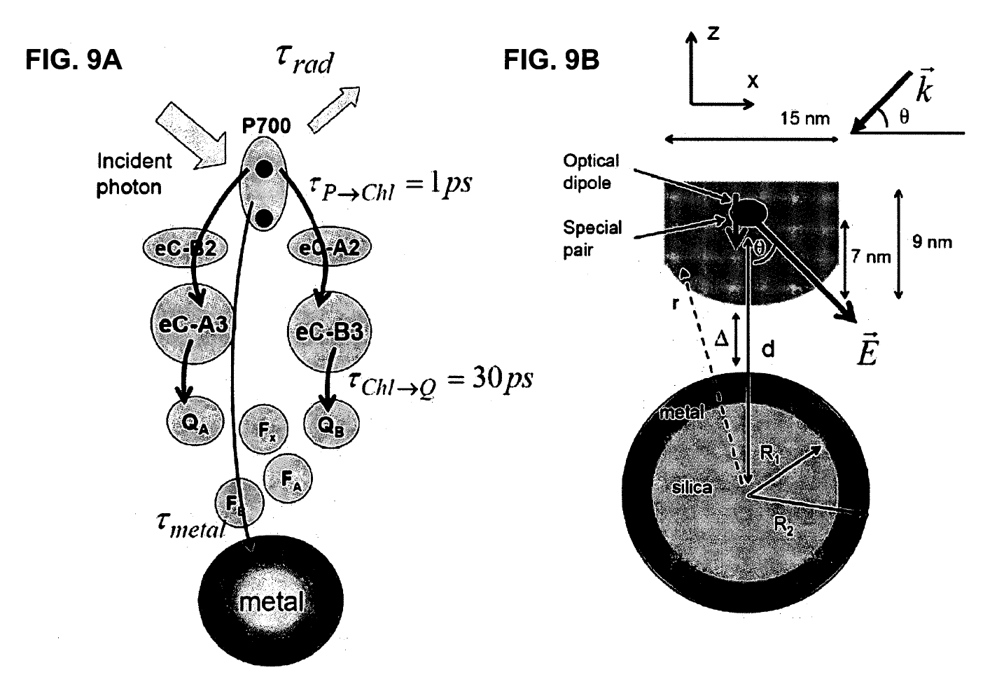

Model of Hybrid Photosystem: The present embodiment incorporates a photosystem I (PS I) reaction center and a single metal NP (FIG. 9). The PS I reaction center from cyanobacteria (Synechocystis sp. PCC6803) is conjugated with a metal NP through a biolinker. The PS I reaction center is composed of the following elements/cofactors: a chlorophyll dimer (special pair, P), two pairs of chlorophylls (eC-B2/eC-A3 and eC-A2/eC-B3), two quinone molecules (QA(B)), and the iron-sulfur centers (F). These building blocks (cofactors) are typical for the reaction centers of many light-harvesting biological systems. In this embodiment, it was assumed that a NP is attached to the iron-centers side (electron donor side) of the reaction center. In principle, a NP can be bound to both sides (acceptor or donor). According to one embodiment, attachment of a NP to the protein can be effected by genetically engineering a cysteine mutant which binds covalently via a sulfide bond to the NP.

Upon absorption of a photon, the special pair P is excited to its higher energy singlet state P*, which transfers an electron along the chains to the chlorophyll cofactors eC−A3/eC−B3 in about τ_{P→Chl}≈1 ps. The chlorophyll cofactors eC−A2/eC−B2 are involved in this initial charge separation process. After fast charge separation, the radiative recombination of the electron-hole pair becomes very unlike. In the next step, the complex P⁺eC−B3⁻/P⁺eC−A3⁻ transfers an electron to the quinone QB⁻/QA⁻ in about in about τ_{Chl→Q}=30 ps. Here Q_{A(B)}⁻ denotes a quinone with one extra electron. This final excited state of the system can be noted as P⁺Q_{A(B)}⁻. After the promotion of Q_{A(B)} into its excited state (≈1.2 eV), the quinones transfer electrons to the iron-sulfur clusters in 250 ns to form the excited state P⁺/F⁻. Since the electron transfer from the special pair to the iron-sulfur clusters is a unidirectional process with almost no losses, the rate of generation of excited quinone molecules Q_{A}⁻ and Q_{B}⁻ will be computed. The present derivations do not depend on the number of active branches since the total number of excited quinone molecules Q⁻=Q_{A}⁻Q_{B}⁻ will be calculated. In the linear regime, the rate equation for the special pair takes a form:

$$\frac{d {n}_{{P}^{*}}}{d t} = α \cdot I - \frac{{n}_{{P}^{*}}}{{τ}_{P -> Chl}} - \frac{{n}_{{P}^{*}}}{{τ}_{rec}} , \qquad ( 1 )$$

where n_{P}* is the average number of excited states of P, n_{P}*<<1, α and I are the absorption coefficient and the light intensity at the special pair, respectively. The rate of recombination is 1/τ_{rec}=1/τ₀+1/τₘₑₜₐₗ, where τ₀ is the time describing the intrinsic losses from the excited state P* inside the reaction center and τₘₑₜₐₗ is the time to transfer of an exciton from P* to the metal NP. The time τₘₑₜₐₗ can become important if the metal NP is located in the very close proximity of P*. It is noted that eq. 1 describes the unidirectional process from P* to P⁺Q⁻ since electron transfer occurs with significant reduction of energy (1.8 eV→1.2 eV). In the stationary regime, the time-derivative in eq. 1 is zero and we obtain: n_{P}*=α·I/γₜₒₜ, where γₜₒₜ=1/τ_{P→Chl}+1/τ_{rec}. From similar equations for the numbers of excited chlorophylls (P⁺eC−B₃⁻ and P⁺eC−A₃⁻) and Q⁻, the rate of Q⁻-generation is obtained:

$${R}_{{Q}_{A}^{-}} = \frac{{n}_{{P}^{+} {Chl}^{-}}}{{τ}_{Clh -> Q}} = \frac{α \cdot I}{1 + {τ}_{P -> Chl} / {τ}_{rec}} ,$$

where n_{P}_{⁺}_{Chl}_{^{−}} is the number of P⁺Chl⁻ complexes. This equation is valid for the linear regime. The quantum yield of the charge separation process is given by

$$Y = \frac{{R}_{{Q}^{-}}}{α \cdot I} = \frac{1}{1 + {τ}_{P -> Chl} / {τ}_{rec}} = \frac{1}{1 + {τ}_{P -> Chl} / {τ}_{0} + {τ}_{P -> Chl} / {τ}_{metal}} . \qquad ( 2 )$$

The light intensity I∝E², where E is the amplitude of the incident electromagnetic field. In the presence of metal NP, this amplitude can be strongly changed due to the induced surface charges. The corresponding enhancement factor is defined as

$$P ( ω ) = \frac{{E}_{z}^{2}}{{E}_{z , no metal}^{2}} , \qquad ( 3 )$$

where E_{z,no metal} is the z-component of the electric field at the P cofactor in the absence of metal NP and E_{Z} is the amplitude of the actual electric field inside the hybrid PS-metal system. Only the z-components are considered here since the optical dipole moment of the reaction center is parallel to the z-axis [Frolov, L.; Rosenwaks, Y.; Carmeli, C.; Carmeli, I. Adv. Mat. 2005, 17, 2434]. Correspondingly, the absorption coefficient strongly depends on the incidence angle: α=α₀ cos θ² (FIG. 9B). Then, the rate becomes:

$${R}_{{Q}^{-}} = \frac{{α}_{0} \cdot {I}_{0} \cdot P ( ω ) \cdot cos {θ}^{2}}{1 + {τ}_{P -> Chl} / {τ}_{0} + {τ}_{P -> Chl} / {τ}_{metal}} , \qquad ( 4 )$$

where I₀∝E_{no metal}².

The present embodiment assumes that the image charges of metal NP do not influence the charge transfer process within the PS complex. This approximation can be justified by estimating the energy of interaction between the photo-excited electron and the induced dipole moment of a NP. The energy of charge-dipole interaction for the parameters of the present model is about 0.05-0.1 eV, whereas the reduction of energy of electron during the transfer process is 0.6 eV. Therefore, it is very unlikely that the transfer process will be affected by the electron-NP interaction.

Plasmon effects: This section focuses on calculation of the plasmon enhancement factor P(ω). The electric potential induced by the incident light in the system shown in FIG. 9B has a form:

$$ϕ = - {E}_{0} r + β ( ω ) \frac{{E}_{0} r}{{r}^{3}} ,$$

where E_{no metal}=E₀e^{-iωt} is the laser field and E₀ is its amplitude; r is the radius vector with respect to the center of NP. The function β(ω) describes the induced dipole moment of NP and depends on the NP geometry and dielectric constant. For spherical NP and NS, this function can be calculated from the quasi-static equation ∇∈(r)∇φ=0 where ∈(r) is the spatially dependent dielectric constant. The coefficients β(ω) for NP and NS are, respectively:

$${β}_{NP} = {R}^{3} \frac{{ɛ}_{m} - {ɛ}_{0}}{( 2 {ɛ}_{0} + {ɛ}_{m} )} , \\ {β}_{NS} = \frac{{R}_{2}^{3} [ {R}_{1}^{3} ( {ɛ}_{d} - {ɛ}_{m} ) ( {ɛ}_{0} + 2 {ɛ}_{m} ) - {R}_{2}^{3} ( {ɛ}_{0} - {ɛ}_{m} ) ( {ɛ}_{d} + 2 {ɛ}_{m} ) ]}{2 {R}_{1}^{3} ( {ɛ}_{0} - {ɛ}_{m} ) ( - {ɛ}_{d} + {ɛ}_{m} ) + {R}_{2}^{3} ( 2 {ɛ}_{0} + {ɛ}_{m} ) ( {ɛ}_{d} + 2 {ɛ}_{m} )} ,$$

where R is the NP radius, and R₁₍₂₎ are the inner (core) and outer (overall) radii of the NS, respectively. The parameters ∈₀, ∈ₘ and ∈_{d} denote the dielectric constants of matrix, metal, and dielectric core of the NS, respectively. The resultant electric field is given by \vec{E}=\vec{E}₀+[3(\vec{d}·\vec{n})\vec{n}−\vec{d}]/r³, where \vec{d}=β\vec{E}₀. Then, the enhancement factor takes the form:

$$P ( ω ) = {\vert 1 + \frac{2 β}{{R}_{NP - dipole}^{3}} \vert}^{2} . \qquad ( 5 )$$

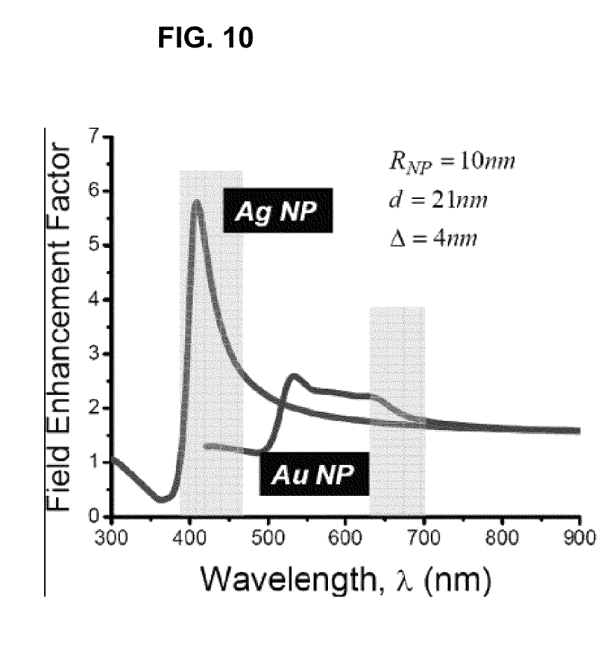

FIGS. 10 and 11 show calculated enhancement factors for the present geometry. In the present calculations, ∈₀=∈_{d}=2.2 is used; this value for ∈_{d} corresponds to the dielectric constant of silica. We also take the matrix dielectric constant ∈₀ slightly increased from the water value (∈_{w}=1.8), to account for the presence of biological molecules. The condition for the plasmon enhancement inside the system is the following:

ω_{abs}≈ω_{plasmon peak},

where _{plasmon peak} and ω_{abs} are the plasmon peak energy of NP/NS and the absorption peak energy of PS system, respectively. The PS reaction center has maxima in the absorption spectrum at ω_{abs}≈1.83 and 2.83 eV (λ≈673 and 436 nm). Grey zones in FIGS. 10 and 11 correspond to the wavevector intervals where this particular reaction center [Frolov, L.; Rosenwaks, Y.; Carmeli, C.; Carmeli, I. Adv. Mat. 2005, 17, 2434] absorbs photons. Other bacterial and plant systems have similar regions of absorption of sun radiation. FIGS. 10 and 11 show that significant enhancement can be achieved for a silver NP at about 436 nm and for both gold and silver nano-shells at about 673 nm.

The calculated plasmon resonances in single Au and Ag spherical nanoparticles lie at about 530 and 420 nm, correspondingly. Since the absorption wavelengths of PS I reaction center are at approximately 436 and 673 nm, the Au NPs are not suitable for the plasmon enhancement effect. However, single Ag NPs can be used. Recently, it was suggested that nano-shells (NSs) can be successfully used to shift plasmon resonances to the red [Halas, N. MRS Bulletin 2005, 30, 362]. In addition to the plasmon shift effect, a stronger enhancement effect can be seen for an NS, compared to that of a NP (compare FIGS. 10 and 11). The maximum enhancement factors for an Ag NP (at about 420 nm), Ag NS (at about 670 nm), and Au NS (680 nm) are the following 6, 15, 10, correspondingly. Especially Ag NSs demonstrate remarkable enhancement for P(ω) and for the corresponding photon-absorption rate α₀·I₀·P(ω). Relatively strong enhancement can be achieved by using Ag NPs since Ag has stronger plasmon resonances. This was also noticed in Lee, J.; Govorov; A. O.; Kotov, N. A. Angew. Chem. 2005, 117, 7605. Lee, J.; Govorov; A. O.; Dulka, J.; Kotov, N. A. Nano Lett. 2004, 4, 2323 and Jiang Jiang, Bosnick, K.; Maillard, M.; Brus, L. J. Phys. Chem. B 2003, 107, 9964. From first look, one can expect appearance of splitting in the plasmon spectrum P(ω) due to the interaction between two plasmons localized near the inner and other surfaces of the NS [Halas, N. MRS Bulletin 2005, 30, 362]. However, in the present example, this splitting is not seen. The reason for this is that the present inventors use empirical dielectrics functions for Au and Ag which give strong broadening to plasmon resonances and also include inter-band transitions. It is easy to see that, for the Drude dielectric function with a small plasmon broadening, the function P(ω) clearly demonstrates the splitting of the plasmon peak.

The enhancement factor was calculated above for the z-component of electric field because the absorbing dipole moment is assumed to be in the z-direction. For the present geometry (FIG. 9B), it means that the dipole moment is perpendicular to the surface of the NP. This configuration is most advantageous for the plasmon enhancement effect [Govorov, A. O.; Bryant, G. W.; Zhang, W.; Skeini, T.; Lee, J.; Kotov, N. A.; Slocik, J. M.; Naik, R. R. Nano Lett. 2006, 6, 984]. For example, if the optical dipole is parallel to the NP surface the factor P(ω) can be either suppressed (NP) or slightly enhanced (NS). For estimations, one can use the equation:

$${P}_{x} ( ω ) = \frac{{E}_{x}^{2}}{{E}_{x , no metal}^{2}} = {\vert 1 - \frac{β}{{R}_{NP - dipole}^{3}} \vert}^{2} .$$

Now, the effect of metal NPs/NS on the quantum yield of quinone production is discussed. To calculate this the fluctuation-dissipation theorem is used and the method developed in Govorov, A. O.; Bryant, G. W.; Zhang, W.; Skeini, T.; Lee, J.; Kotov, N. A.; Slocik, J. M.; Naik, R. R. Nano Lett. 2006, 6, 984. The energy transfer time for a dipole is given by 1/τₘₑₜₐₗ(ω)=−2/ImF(ω), where F(ω) is the response function. In the dipole limit (d>R_{NP}, R₁), we can obtain analytical results (for details see Govorov, A. O.; Bryant, G. W.; Zhang, W.; Skeini, T.; Lee, J.; Kotov, N. A.; Slocik, J. M.; Naik, R. R. Nano Lett. 2006, 6, 984)

$$\frac{1}{{τ}_{metal , NP / NS} ( ω )} = - \frac{8}{ℏ} \frac{{e}^{2} \cdot {d}_{sp}^{2}}{{d}^{6} {ɛ}_{0}^{2}} \frac{Im [ {ɛ}_{m} ( ω ) ]}{4 π} {f}_{NP / NS} ( ω ) , \\ {f}_{NP} ( ω ) = \frac{4 π}{3} {R}_{NP}^{3} {\vert \frac{3 {ɛ}_{0}}{{ɛ}_{m} ( ω ) + 2 {ɛ}_{0}} \vert}^{2} . \qquad ( 6 )$$

For the NS case, the function f_{NS}(ω) can be written as:

$${f}_{NS} = \frac{4}{105} π ( \begin{matrix}122 \cdot {C}_{1} \cdot {C}_{2}^{*} \{ \frac{1}{{R}_{1}^{3}} - \frac{1}{{R}_{2}^{3}} \} + 35 \cdot {B}_{1} \cdot {B}_{2}^{*} \{ - {R}_{1}^{3} + {R}_{2}^{3} \} + \\ 105 \cdot \{ {B}_{2}^{*} \cdot {C}_{1} + {B}_{1} \cdot {C}_{2}^{*} \} \cdot \{ Log [ {R}_{1} ] - Log [ {R}_{2} ] \}\end{matrix} )$$

$${B}_{1} = 3 \{ \begin{matrix}{R}_{2}^{3} \cdot {ɛ}_{0} \cdot {ɛ}_{d} + \\ 2 {R}_{2}^{3} \cdot {ɛ}_{0} \cdot {ɛ}_{m}\end{matrix} \} / ( \begin{matrix}- 2 {R}_{1}^{3} \cdot {ɛ}_{0} \cdot {ɛ}_{d} + 2 {R}_{2}^{3} \cdot {ɛ}_{0} \cdot {ɛ}_{d} + 2 {R}_{1}^{3} \cdot {ɛ}_{0} \cdot {ɛ}_{m} + 4 {R}_{2}^{3} \cdot {ɛ}_{0} \cdot {ɛ}_{m} + \\ 2 {R}_{1}^{3} \cdot {ɛ}_{d} \cdot {ɛ}_{m} + 2 {R}_{2}^{3} \cdot {ɛ}_{d} \cdot {ɛ}_{m} - 2 {R}_{1}^{3} \cdot {ɛ}_{m}^{2} + 2 {R}_{2}^{3} \cdot {ɛ}_{m}^{2}\end{matrix} )$$

$${C}_{1} = - 3 \{ \begin{matrix}- {R}_{1}^{3} \cdot {R}_{2}^{3} {ɛ}_{0} \cdot {ɛ}_{d} + \\ {R}_{1}^{3} \cdot {R}_{2}^{3} {ɛ}_{0} \cdot {ɛ}_{m}\end{matrix} \} / ( \begin{matrix}\begin{matrix}- 2 {R}_{1}^{3} \cdot {ɛ}_{0} \cdot {ɛ}_{d} + 2 {R}_{2}^{3} \cdot {ɛ}_{0} \cdot {ɛ}_{d} + \\ 2 {R}_{1}^{3} \cdot {ɛ}_{0} \cdot {ɛ}_{m} + 4 {R}_{2}^{3} \cdot {ɛ}_{0} \cdot {ɛ}_{m} +\end{matrix} \\ \begin{matrix}2 {R}_{1}^{3} \cdot {ɛ}_{d} \cdot {ɛ}_{m} + 2 {R}_{2}^{3} \cdot {ɛ}_{d} \cdot {ɛ}_{m} - \\ 2 {R}_{1}^{3} \cdot {ɛ}_{m}^{2} + 2 {R}_{2}^{3} \cdot {ɛ}_{m}^{2}\end{matrix}\end{matrix} )$$

$${B}_{2} = 3 \{ \begin{matrix}{R}_{2}^{3} \cdot {ɛ}_{0} \cdot {ɛ}_{d} + \\ 2 {R}_{2}^{3} \cdot {ɛ}_{0} \cdot {ɛ}_{m}\end{matrix} \} / ( \begin{matrix}- 2 {R}_{1}^{3} \cdot {ɛ}_{0} \cdot {ɛ}_{d} + 2 {R}_{2}^{3} \cdot {ɛ}_{0} \cdot {ɛ}_{d} + 2 {R}_{1}^{3} \cdot {ɛ}_{0} \cdot {ɛ}_{m} + 4 {R}_{2}^{3} \cdot {ɛ}_{0} \cdot {ɛ}_{m} + \\ 2 {R}_{1}^{3} \cdot {ɛ}_{d} \cdot {ɛ}_{m} + 2 {R}_{2}^{3} \cdot {ɛ}_{d} \cdot {ɛ}_{m} - 2 {R}_{1}^{3} \cdot {ɛ}_{m}^{3} + 2 {R}_{2}^{3} \cdot {ɛ}_{m}^{2}\end{matrix} )$$

$${C}_{2} = - 3 \{ \begin{matrix}- {R}_{1}^{3} \cdot {R}_{2}^{3} {ɛ}_{0} \cdot {ɛ}_{d} + \\ {R}_{1}^{3} \cdot {R}_{2}^{3} {ɛ}_{0} \cdot {ɛ}_{m}\end{matrix} \} / ( \begin{matrix}\begin{matrix}- 2 {R}_{1}^{3} \cdot {ɛ}_{0} \cdot {ɛ}_{d} + 2 {R}_{2}^{3} \cdot {ɛ}_{0} \cdot {ɛ}_{d} + \\ 2 {R}_{1}^{3} \cdot {ɛ}_{0} \cdot {ɛ}_{m} + 4 {R}_{2}^{3} \cdot {ɛ}_{0} \cdot {ɛ}_{m} +\end{matrix} \\ \begin{matrix}2 {R}_{1}^{3} \cdot {ɛ}_{d} \cdot {ɛ}_{m} + 2 {R}_{2}^{3} \cdot {ɛ}_{d} \cdot {ɛ}_{m} - \\ 2 {R}_{1}^{3} \cdot {ɛ}_{m}^{2} + 2 {R}_{2}^{3} \cdot {ɛ}_{m}^{2}\end{matrix}\end{matrix} )$$

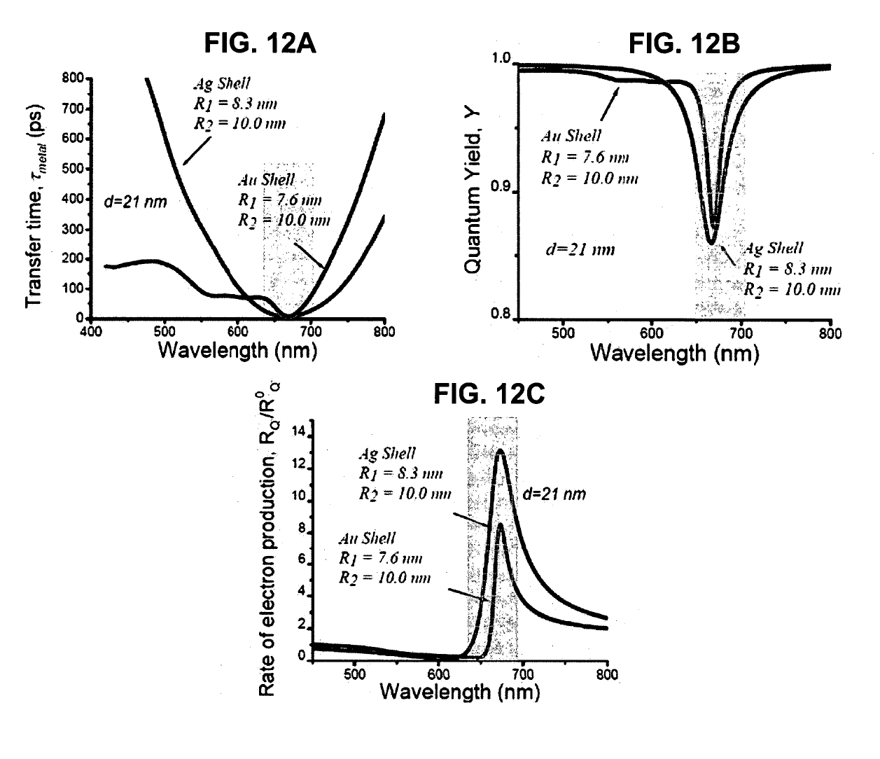

Equation (6) above describes the energy transfer from a z-oriented dipole. The transfer times depend on the dipole moment of the special pair dₛₚ. To estimate this parameter, the expression for a molecular radiative lifetime is used [Yariv, A. Quantum Electronics, 2^{nd} Ed., New York, John Wiley & Sons, 1975]

$$1 / {τ}_{rad} = \frac{8 π \sqrt{{ɛ}_{0}} {ω}_{exc}^{3} {e}^{2} {d}_{sp}^{2}}{3 h \cdot {c}^{3}} ,$$

where ω_{exc} is the exciton energy of the special pair. With typical molecular radiative lifetime τ_{rad}≈5 ns and ω_{exc}≈1.8 eV, dₛₚ≈0.26 nm is obtained. The above value is a typical number for molecular systems. First we calculate the transfer times for Au and Ag nano-shells. In the absence of the metal subsystem, the quantum yield is close to unit. Taking τ_{rad}≈5 ns and τ_{P→BPh}=4 ps we have Y₀≈0.999. FIG. 12 shows the transfer times τₘₑₜₐₗ, quantum yield Y and the relative quinone production rates:

$$\frac{{R}_{{Q}^{-}}}{{R}_{{Q}^{-}}^{0}} = P ( ω ) \frac{Y}{{Y}^{0}} = P ( ω ) \frac{1 + {τ}_{P \to Chl} / {τ}_{0}}{1 + {τ}_{P \to Chl} / {τ}_{0} + {τ}_{P \to Chl} / {τ}_{metal}} .$$

Here R_{Q}⁰- and Y⁰ are the corresponding values in the absence of the metal subsystem. In FIG. 12, a remarkable enhancement of the production rate

$$\frac{{R}_{{Q}^{-}}}{{R}_{{Q}^{-}}^{0}}$$

is seen. As was expected, the quantum yield of the PS reaction center becomes reduced due to the energy transfer to the metal. However, this decrease is significantly less than the field enhancement P(ω). Both quantities Y(ω) and P(ω) become strongly changed in the vicinity of the plasmon resonance. The minimum of Y(ω) is slightly to the blue compared to the maximum of P(ω). This behavior makes the enhancement effect even stronger.

Equations. (2) and (4) describing the plasmon effects has two important parameters: enhancement factor P(ω) and energy dissipation rate 1/τₘₑₜₐₗ. The net effect of the production rate depends on competition between the above parameters. The same interplay of field enhancement and energy dissipation was found for the emission process of semiconductor NPs in the vicinity of the metal nanocrystals [Lee, J.; Govorov; A. O.; Kotov, N. A. Angew. Chem. 2005, 117, 7605. Lee, J.; Govorov; A. O.; Dulka, J.; Kotov, N. A. Nano Lett. 2004, 4, 2323; Govorov, A. O.; Bryant, G. W.; Zhang, W.; Skeini, T.; Lee, J.; Kotov, N. A.; Slocik, J. M.; Naik, R. R. Nano Lett. 2006, 6, 984]. The parameters P(ω) and 1/τₘₑₜₐₗ strongly depend on the geometry, dipole orientation, and NP material. Therefore, the design of hybrid structures and the understanding of physical mechanisms can be crucial.

In the case of Ag NP, the plasmon enhancement effect is expected at about 430 nm (FIG. 10). In this case, the exciton in the special pair is created indirectly. First the photon creates the excitation with energy 2.83 eV (λ≈436 nm). Then, the system makes transition to the state with energy 1.83 eV (λ≈673 nm). Using the rate equation we obtain the quantum yield:

Y(ω)≈1−(τ_{exc→P}*/τ_{0,exc}+τ_{exc→P}*/τ_{metal, 2.83eV}(ω)+τ_{P→Chl}/τ_{0,P}*+τ_{P→Chl}/τ_{metal,1.83eV})

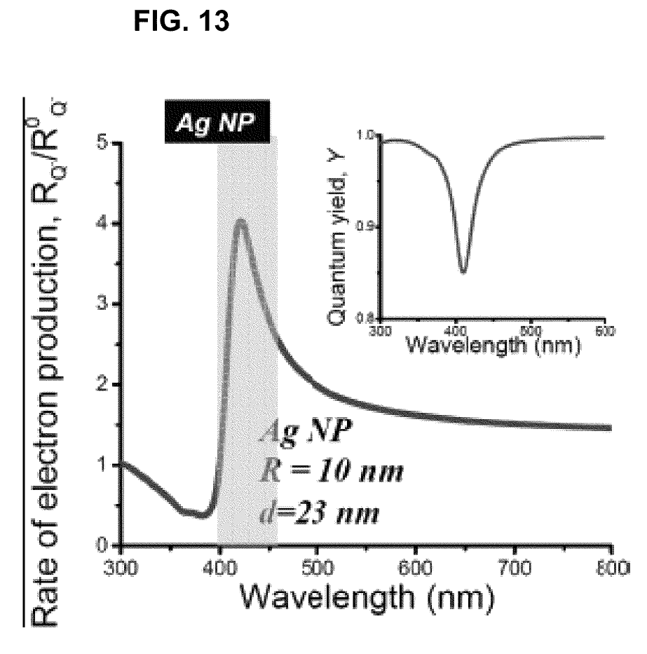

, where τ_{metal,1.83eV} is the PS-metal transfer time at the specified energy and τ_{exc→P}* is the relaxation time from the exciton to the state P* with the exciton energy of 1.83 eV. For the time τ_{exc→P}*, we took 2 ps. The above simplified equation is valid if the transfer times τₘₑₜₐₗ>>τ_{P→BPh}, τ_{exc→P}*. FIG. 13 summarizes the results for the Ag NP. Again significant enhancement of the normalized quinone production rate

$$\frac{{R}_{{Q}_{A}^{-}}}{{R}_{{Q}_{A}^{-}}^{0}}$$

is observed. Of note, the energy transfer times in the regime of small d (d≈R,R₁₍₂₎) should be calculated numerically. One convenient numerical method is described in Govorov, A. O.; Bryant, G. W.; Zhang, W.; Skeini, T.; Lee, J.; Kotov, N. A.; Slocik, J. M.; Naik, R. R. Nano Lett. 2006, 6, 984; it incorporates the multi-pole expansion approach and fluctuation-dissipation theorem.

Discussion

An advantage of the hybrid nanostructure of the present embodiments is that amount of chemical energy (number of excited electrons) per reaction center is significantly increased. In one monolayer of reaction centers, the total absorption can be relatively small but the excited-quinone production in the presence of metal NP/NS is strongly increased by factor of 5-15. If larger metal NP are chosen or a special NP complex is designed, the enhancement factor of electron production RQ-/R⁰Q- can be further increased. The PS-NP complexes can be studied in solution (a) as a monolayer bound to a metal surface (b), and/or inside biological membranes (c). In the case (a), one can study the effect of plasmon-enhanced absorption by a photosynthetic complex. In the case (b), a monolayer of hybrid nanostructure can be placed between electrically conductive surfaces and photocurrent can be studied. In nature, bacterial and other reaction centers are built in a membrane and surrounded by the antenna chlorophylls. These chlorophylls absorb photons and transfer optically created excitons toward the reaction center. With metal NP, one can also enhance optical absorption of the antenna chlorophylls. For efficient light harvesting, the Forster transfer times between chlorophylls and charge separation time are preferably smaller than the time of transfer to metal.

Enhanced generation of excited electrons appear due to increased absorption and very fast change separation within a photosynthetic molecule. The photogenerated electron and hole become spatially separated very fast, within about 1 ps. This time is shorter than the typical times to transfer exciton energy to a metal NP. This inequality (τ_{P→Chl}<<τₘₑₜₐₗ) ensures that the energy losses are relatively small. It is noted that a photosynthetic system with fast spatial charge separation resembles photodiodes and type-II semiconductor heterostructures in which photogenerated electron-hole pairs became rapidly separated in space due to the built-in potential. Compared to solid-state devices, advantages of the nanostructure of the present embodiments are in three-dimensional architecture, self-assembly and photochemical responses.

The present example demonstrates that a hybrid nanostructure composed of photosynthetic molecules and metal nanoparticles and nanoshells can greatly enhance photo-chemical production or photocurrents, despite the reduced quantum yield.

### Example 3

### Supporting Data for Hybrid Metal Nano Particles Ps I

As shown in Example 2, the efficiency of chemical energy production PS I is strongly enhanced in the presence of metal nanoparticles (NP). In the case of photo-transport experiments with the photosynthetic reaction centers, the plasmon resonance generated in the NP enhances the chlorophyll's absorption and increases the photocurrent response in PS I. The type of metal used and the size of the NP can be tuned to generate plasmon with energy that can efficiently enhance the absorption by PS I. For example, the present inventors calculated that gold or silver coated silicon nanoparticles of 21 nm in diameter will generate plasmon resonance frequency that enhance the absorption of light by PS I at peak absorption at about 700 nm. Plasmons are tuned to the energy that overlaps the absorption of PSI to efficiently enhance absorption at the two absorption maxima of PS I. The present inventors also calculated that light energy can enhance electron generation in the PS I hybrid gold NP and silver NP by factors of 10 and 15 fold, respectively. By tuning the total size and the size of the coated layer other metals can be used to generate plasmons with energy that can efficiently effect the pigments and enhance the efficient charge separation process and the current generated by PS I in future optoelectronic devices.

Materials and Methods

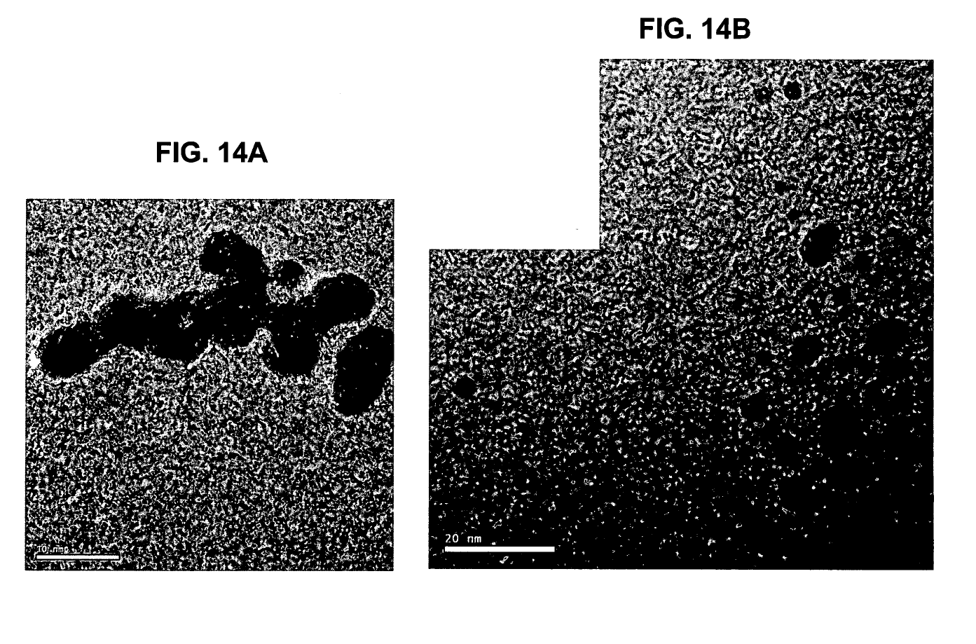

Fabrication of NP/PSI hybrids. Hybrid metal NP PS I are fabricated by direct covalent binding between NP and PSI. Cysteine mutants at the oxidizing end of PS I are bound to the metal NP by formation of sulfide bond between the cysteine thiols and the metal. NP are fabricated by reduction of 1 mM solution of AuCl₃ and AgNO₃ by BH₄ in the presence of 5 nM PSI. Hybrid PSI/NP made of Au and Ag of approximately 5 nm are fabricated as can be seen by electron microscopy (FIGS. 14A-B).

Results

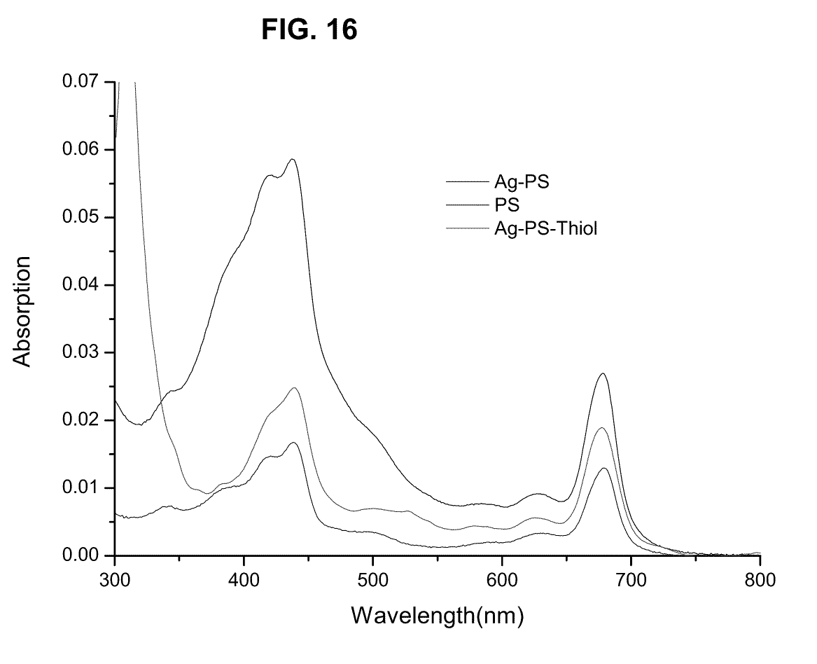

Enhanced absorption in the AuNP/PSI and AgNP/PSI hybrids. The absorption spectra of PSI (blue line) with peak absorption at 438 nm and 678 nm is enhanced by ˜5 folds in the AuNP/PSI hybrids (black line) (FIG. 15). The Au NP plasmon is shown as a broad band absorption maximum around 550 nm. Addition of excess thioglycolate which partially dissociate the NP PSI bonds reduce the enhancement (red) indicating that a tight covalent binding between PSI and the NP contributes to the efficiency of plasmon enhancement. This is proof of the concept that was developed in the theoretical calculations presented in Example 2, according to which tuning of the NP size should induce optimal plasmon enhancement of PSI absorption. Reduction of Ag⁺⁺ ions in the presence of PSI produces AgNP/PSI hybrids of approximately 5 nm in size as can be seen in the electron microscopy images (FIG. 14A). The absorption spectra of PSI (blue line) with peak absorption at 438 nm and 678 nm is enhanced by ˜3.7 and ˜2.14 folds in the AuNP/PSI hybrids (black line) (FIG. 16). Addition of excess thioglycolate which partially dissociate the NP PSI bonds reduce the enhancement (red) indicating that a tight covalent binding between PSI and the NP contributes to the efficiency of plasmon enhancement.

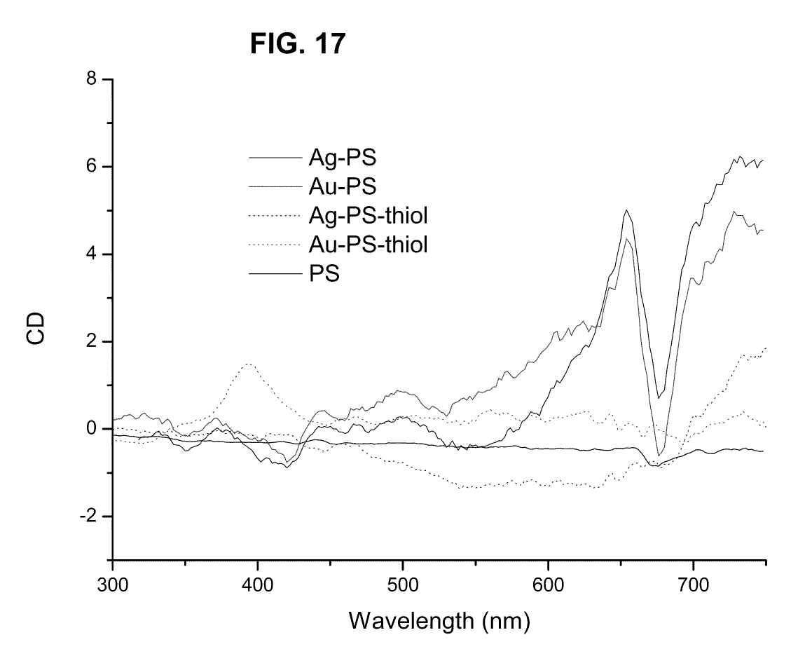

Enhanced circular dicroism (CD) spectra of AuNP/PSI and AgNP/PSI hybrids. The circular dicroism (CD) spectra of the chlorophylls in the NP/PSI hybrids is more sensitive than the absorption spectra to plasmon enhancement. The plasmon has no signal in the visible region of the CD spectra and therefore the spectra of the NP/PSI hybrid is free from the contribution of the plasmon to the total signal. A 10 fold plasmon enhancement is clearly seen when the CD spectra of PSI (black) is compared to the spectra of AuNP/PSI (red) and AgNP/PSI (blue) hybrids (FIG. 17). The enhancement effect is more sensitive to the distance between the NP and PSI as the detachment of the sulfide bond between the NP and PSI on addition of thioglycolate (red and blue dots) completely reverses the plasmon enhancement (FIG. 17).

### Example 4

### Enhanced Optical Properties of a Photosynthetic System Conjugated with Semiconductor Nanoparticles: The Role of Förster Transfer

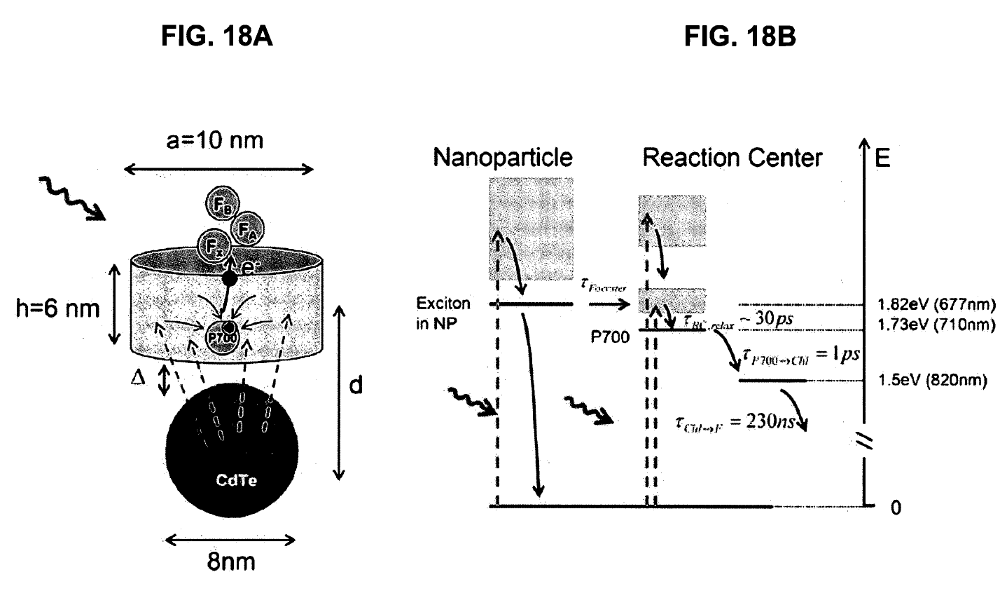

The present embodiment comprises a nanostructure which incorporates a photosystem I (PS I) and one or more semiconductor NPs, which are connected to the PS I by a bio-linker (FIG. 18A-B). For clarity of presentation, the following description is provided for the case in which the nanostructure includes one semiconductor NP which is bound to the PS I. One of ordinary skill in the art would know how to adjust the description for the case of more than one semiconductor NP.

For a photosystem, the PS I reaction center (RC) from cyanobacteria (Synechocystis sp. PCC6803) was selected. In this example, it is assumed that the NP is bound to the RC from the electron donor side. In principle, a NP can be attached to the electron acceptor side or, when more than one NP is employed, to both the acceptor and donor sides. This may be effected using a sulfide bond with cysteine mutant in the protein. In the light-harvesting process, an optically-excited electron-hole pair becomes trapped at the spatial pair. Then, the special pair loses an electron which travels across the membrane along the electron-transfer chain toward the Fe₄S₄ clusters. This electron transfer and sequential excitation of Fe₄S₄ clusters trigger a series of reactions, which eventually result in synthesis of ATP and reduction of NADP⁺.

The spatial structure of a RC of the PS I has been revealed in the X-ray studies. The dimensions of the RC are 10 nm×9 nm. The main body of chlorophyll molecules is found within a volume with dimensions 10 nm×6 nm. Therefore, the present inventors model an ensemble of absorbing chlorophylls as a cylinder of radius 5 nm and height 6 nm (FIG. 18A). The RC contains many chlorophyll molecules with different orientation of dipoles and absorbs photons coming from all directions. Therefore, it may be assumed that absorption of light by “the chlorophyll cylinder” is isotropic. FIG. 18B shows the diagram of flow of energy in the hybrid system. First, incident photons are absorbed by both the NP and RC. Excitons optically generated inside the NP can recombine inside the NP or can be transferred to the RC. The transfer process occurs via the FT mechanism. FT comes from the Coulomb interaction and does not require tunnel coupling between two objects. The excitation in the RC undergoes fast energy relaxation and ends up at the P700 special pair. The time of migration of excitation towards P700 inside the RC is τ_{RC,relax}˜30 ps. Once the excitation becomes trapped at the P700, the electron and hole are very efficiently separated, within τ_{P700→Chl}=1 ps. In this process, the excited electron is transferred to the primary acceptor chlorophylls, whereas the hole remains trapped at the P700. A very high internal quantum yield of a PS I is guaranteed by very fast spatial separation of photo-generated electron and hole at the P700 special pair. The separation time τ_{P700→Chl} is much shorter than the typical recombination lifetimes in molecules (˜1 ns). After electron transfer to the primary acceptor, the electron makes slower transitions and, in 230 ns, ends up in Fe₄S₄ clusters. The above processes are unidirectional since they occur with loss of energy.

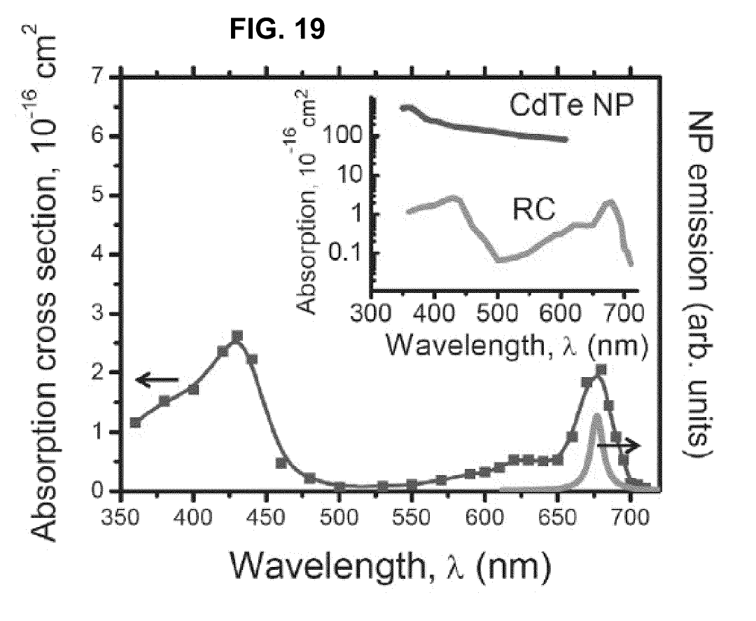

The absorption spectrum of the RC has two bands with wavelengths λ_{RC1}˜680 nm and λ_{RC2}˜430 nm. The 96 Chls inside the RC are responsible for the 680 nm-band. The FT process is effective if the overlap between the emission spectrum of NP and the absorption spectrum of RC is significant. The absorption of optical energy inside the NP occurs if λ<λ_{exc}, where λ and λ_{exc} are the wavelengths of incident light and the exciton emission, respectively. Therefore, in order to assure efficient FT between NP and RC the condition: λ_{RC1}≈λ_{exc} should be satisfied. In this embodiment, a spherical CdTe NP is chosen with a radius of R_{NP}=4 nm. For this NP, the corresponding exciton emission wavelength can be estimated as λ_{exc}=677 nm. This choice of NP parameters generates a strong overlap between the NP and RC spectra (FIG. 19). In the next step, the absorption spectrum of CdTe NP is modeled. In [Leatherdale, W. K. Woo, F. V. Mikulec, M. G. Bawendi, J. Phys. Chem. B 2002, 106, 7619], it has been experimentally shown that the absorption cross-section of colloidal NPs scales as ∝R_{NP}³ if the wavelength of absorbed light is lower than λ_{exc}. It means that, for short wavelengths, the quantization can be neglected and a dielectric model of NP can be used to compute the absorption cross section. Strictly speaking, this approach is valid for the excitation energies: E−E_{exc}>E_{quant}, where E_{exc} and E_{quant} are the exciton and quantization energies, respectively. Within the simplified envelope-function method,

$${E}_{quant} = \frac{{ℏ}^{2} {π}^{2}}{2 μ {R}_{NP}^{2}} and {E}_{exc} = {E}_{g , CdTe} + {E}_{quant} ,$$

where μ=1/(mₑ⁻¹+mₕ⁻¹) is the reduced effective mass of the exciton. A monochromatic electromagnetic wave that creates an electric field \vec{e}E₀ cos ωt in the vicinity of the hybrid structure is now considered. The absorption rate of a spherical NP is given by:

$${Q}_{abs} = \frac{1}{2} Re {\int }_{{V}_{RC}} {\vec{j}}^{*} ( r ) \vec{E} ( r ) d V = {σ}_{NP} {I}_{0} , \qquad ( 1 )$$

where ∈_{NP}=∈_{NP}(ω) is the dynamic dielectric constant of CdTe,

$$\vec{E} ( r ) = \frac{3 {ɛ}_{0}}{2 {ɛ}_{0} + {ɛ}_{NP}} \vec{e} {E}_{0} {e}^{- iω t}$$

is the electric field inside the NP,

$$\vec{j} ( r ) = - iω \frac{{ɛ}_{NP} - 1}{4 π} \vec{E} ( r )$$

is the induced current, and ∈₀ is the dielectric constant of a matrix. ∈₀=1.8 is chosen, which corresponds to water and is also very close to the typical values for proteins (n₀=1.33). The incident light intensity and electric field are related through the equation

$${E}_{0}^{2} = \frac{8 π {I}_{0}}{c \sqrt{{ɛ}_{0}}} .$$

The absorption cross-section of the NP can now be written as:

$${σ}_{NP} ( ω ) = {V}_{NP} \frac{ω}{c \sqrt{{ɛ}_{0}}} {\vert \frac{3 {ɛ}_{0}}{2 {ɛ}_{0} + {ɛ}_{NP}} \vert}^{2} Im {ɛ}_{NP} , \qquad ( 2 )$$

where V_{Np}=4πR_{NP}³/3 is the NP volume. In practice, Equation 2 provides a reliable estimate for λ_{exc}−λ≦50 nm. For example, the calculated cross section for a CdSe NP with R_{NP}=3.4 nm at λ=500 nm is 10·10⁻¹⁵ cm² and the corresponding experimental value obtained in Leatherdale, W.-K. Woo, F. V. Mikulec, M. G. Bawendi, J. Phys. Chem. B 2002, 106, 7619 is 8.4·10⁻¹⁵ cm². In the inset of FIG. 19, the NP absorption cross section calculated for CdTe NP [E. D. Palik, Handbook of Optical Constants of Solids; Academic Press: New York, 1985] is compared with the absorption by RC. The results show that σ_{NP}>>_{RC}; this inequality is typical for molecular and crystalline systems. This strongly suggests that one can take advantage by designing a hybrid system, in which the semiconductor component has a very large absorption cross section and the bio-molecular component has an important functionality (such as efficient separation of electron-hole pair in the RC).

In the present model, the RC is approximated as an absorbing cylinder with a local isotropic conductivity β₀(ω). Inside the RC cylinder, the incident electromagnetic wave induces dissipative currents \vec{j}=β₀(ω)\vec{E}. Then, it can be obtained from Equation (1):

$$Re {β}_{0} = {σ}_{RC} \frac{c \sqrt{{ɛ}_{0}}}{4 π {V}_{RC}} ,$$

where σ_{RC} is the absorption cross section of RC (see FIG. 19) and V_{RC}=πα² h/4 is the RC volume.

Exciton dynamics inside the hybrid nanostructure is given by the standard rate equations:

$$\frac{\partial {n}_{NP}}{\partial t} = \frac{{σ}_{NP} {I}_{0}}{ℏω} - ( {γ}_{NP}^{0} + {γ}_{Foerster} ) {n}_{NP} , \\ \frac{\partial {n}_{P 700}}{\partial t} = \frac{{σ}_{RC} {I}_{0}}{ℏω} + {γ}_{Foerster} {n}_{NP} - \frac{{n}_{P 700}}{{τ}_{P 700 \to Chl}} , \qquad ( 3 )$$

where n_{NP} and n_{P700} are the averaged numbers of excitons in a NP and at the P700 special pair; γ_{NP}⁰=1/τ_{rec}⁰ and γ_{Foerster} are the intrinsic recombination rate of a NP and the rate of FT from a NP to a RC, respectively. Equations (3) assume that the intra-RC relaxation time τ_{RC,relax} is much shorter than the FT time. Indeed, τ_{RC,relax}˜30 ps and 1/γ_{Foerster} will be found in the range of 40 ns. Due to the inequality τ_{RC,relax}<<1/γ_{Foesrter}, the population of excited states of a RC does not enter Eq. 3 explicitly. Under the stationary conditions

$$( \frac{\partial {n}_{P 700}}{\partial t} = 0 and \frac{\partial {n}_{P 700}}{\partial t} = 0 )$$

the rate of generation of excited electrons in the electron-transfer chain calculated for the hybrid complex is given by:

$${R}_{HS} = \frac{{n}_{P 700}}{{τ}_{P 700 \to Chl}} = \frac{{I}_{0}}{ℏω} ( {σ}_{RC} + {σ}_{NP} \frac{{γ}_{Foerster}}{( {γ}_{NP}^{0} + {γ}_{Foerster} )} ) . \qquad ( 4 )$$

The rate γ_{Foerster} describes the strength of interaction between the NP and RC. A spherical CdTe NP has three optically-active excitons with dipole moments along the x-, y-, and z-directions. Since the spin lifetime of an exciton in a NP at room temperature is short, populations of different types of excitons are almost the same. Therefore, the rate of FT in Eq. 4 can be described as an average rate γ_{Foerster}=(γₓ+γ_{y}+γ_{z})/3, where γ_{α} is the rate of transfer for the exciton with a dipole moment along the α-axis (α=x,y,z). A convenient formalism to calculate the FT rates for excitons in NPs is given in A. O. Govorov, G. W. Bryant, W. Zhang, T. Skeini, J. Lee, N. A. Kotov, J. M. Slocik, R. R. Naik, Nano Lett. 2006, 6, 984; A. O. Govorov, A. O., J. Lee, J., Kotov, N. A. Phys. Rev. B 2007, 76, 125308. This FT rate can be computed as

$$\begin{matrix}{γ}_{α} = \frac{2}{ℏω} Re {\int }_{{V}_{RC}} {\vec{j}}^{α} ( r ) \cdot {\vec{E}}^{α *} ( r ) d V \\ = \frac{2}{ℏω} {σ}_{RC} ( {ω}_{exc} ) \frac{c \sqrt{{ɛ}_{0}}}{4 π {V}_{RC}} {\int }_{{V}_{RC}} {\vec{E}}^{α} ( r ) \cdot {\vec{E}}^{α *} ( r ) d V ,\end{matrix} \qquad ( 5 )$$

where \vec{E}^{a} is the electric field induced by an oscillating dipole associated with the α-exciton; its field is given by is

$${\vec{E}}_{α} ( r ) = - \vec{∇} \frac{{ed}_{exc} ( \vec{r} \cdot {\vec{e}}_{α} )}{{{ɛ}_{eff} ( {x}^{2} + {y}^{2} + {z}^{2} )}^{3 / 2}} ,$$

where \vec{r} is the radius vector, d_{exc} is the magnitude of dipole moment, ∈_{eff}=(2∈₀+∈_{CdTe})/3, and \vec{e}_{α} is the unit vector along the dipole; for the dielectric constant of semiconductor, we take ∈_{CdTe}=7.2. In Eq. 5, the electric current is induced by the dipole field of exciton \vec{j}^{α}=β₀(ω)\vec{E}^{α}. In addition, from simple algebra,

$${\sum }_{α = x , y , z} {\vec{E}}^{α} ( r ) \cdot {\vec{E}}^{α *} ( r ) / 3 = 2 / {r}^{6}$$

may be obtained.

Equation 5 was written under the assumption that the exciton in a NP has a definite energy. In reality, the exciton peak is broadened, which results in a replacement in Eq. 5:

$${σ}_{RC} \to {\overline{σ}}_{RC} = {\int }_{{ω}_{min}}^{{ω}_{max}} F ( ω ) {σ}_{RC} ( ω ) d ω ,$$

where the function F(ω) describes the normalized absorption of a donor (i.e. a NP) in the FT process:

$$F ( ω ) = \frac{1}{π} \frac{Δ ω}{{( ω - {ω}_{exc} )}^{2} + Δ {ω}^{2}} .$$

For the exciton broadening of a NP, a typical number Δω=27 meV may be taken that corresponds to the FWHM 2Δλ=20 nm. For the limits in the integral: ωₘᵢₙ₍ₘₐₓ₎=1.73 eV(2.30 eV) may be chosen. The resultant FT rate takes a form

$${γ}_{Foerster} = {\overline{σ}}_{RC} \frac{c \sqrt{{ɛ}_{0}}}{ℏωπ} {( \frac{{ed}_{exc}}{{ɛ}_{eff}} )}^{2} \frac{1}{{d}^{6}} A , \qquad ( 6 )$$

where d is the NP-RC center-to-center distance and

$$A = \frac{{d}^{6}}{{V}_{RC}} {\int }_{{V}_{RC}} \frac{1}{{r}^{6}} d V$$

is a geometrical factor. The exciton dipole can be estimated from a typical exciton lifetime of CdTe NPs. d_{exc}=0.2 nm is obtained, assuming τ_{rec}⁰≈20 ns and the quantum yield Y_{NP}=1. With the geometrical parameters a=10, h=6, R_{NP}=4, and Δ=1 nm, the calculated geometrical factor is A=1.3. The resultant FT rate γ_{Foerster}=2.5·10⁷ s⁻¹. This number implies that the time of energy transfer (τ_{Foerster}=1/γ_{Foerster}) is longer than the exciton recombination time of NP: τ_{Foerster}/τ_{rec}⁰≈2. In FIG. 3, we show the rates of generation of excited electrons for the hybrid NP-RC system (R_{HS}) and for the RC alone (R_{RC}). The rate R_{HS} was calculated from Eq. 4 and R_{RC}=I₀σ_{RC}/ω. A very strong increase of the rate R_{HS} is seen compared to R_{RC}. Interestingly, this happens despite the fact that

$$\frac{{γ}_{Foerster}}{( {γ}_{NP}^{0} + {γ}_{Foerster} )} ∼ 0.33 < 1.$$

The physical reason for the strong enhancement in the hybrid system is the strongly enhanced absorption in a CdTe NP. It is also noted that Eq. 6 takes into account FT from a NP to the surface of the RC. For the chosen parameters, NP-surface transfer leads to a slightly increased geometrical factor (A=1.3). For small exciton-surface distances (R_{NP}+Δ), the geometrical factor A becomes a large number.

The ratio of the total absorption of sunlight for the systems can now be evaluated. The sunlight spectrum is close to the spectrum of black body at Tₛ=5200K,

$${U}_{ω} ∝ {ω}^{3} / ( {e}^{\frac{ℏω}{{k}_{B} {T}_{s}}} - 1 )$$

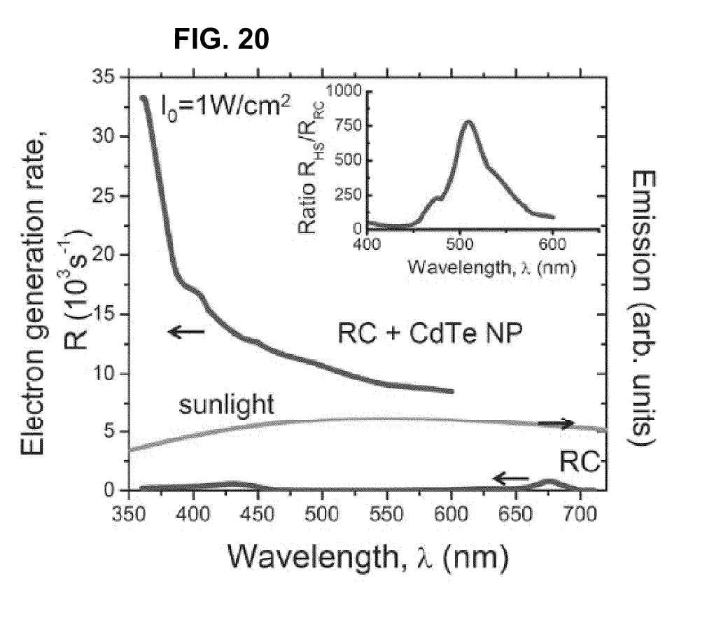

(see FIG. 20). The total rate of electron generation within a given photon-energy interval is given by:

$${R}_{tot , HS} = {\int }_{{ω}_{1}}^{{ω}_{2}} {U}_{ω} \cdot {R}_{HS} ( ω ) d ω .$$

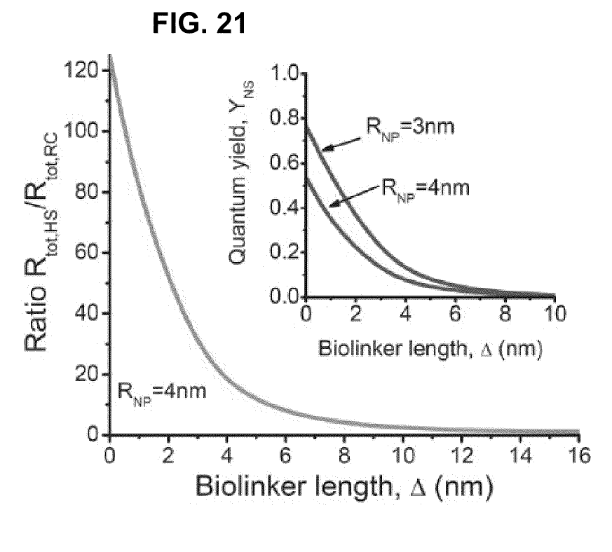

Using Eq. 4 and the experimental data for R_{HS} (ω), we obtain: R_{tot,HS}/R_{tot,RC}≈77. For the limits of integration, we assumed ω₁₍₂₎=2.05 eV(3.42 eV) that corresponds to λ₁₍₂₎=360 nm(600 nm). Within the above interval, the cross section of the NP is well approximated by Eq. 2. In principle, the hybrid NP-RC complex can harvest light energy in the interval ω>ω_{exc}=1.82 eV or λ<λ_{exc}=677 nm. The ratio R_{tot,HS}/R_{tot,RC} strongly depends on the distance between the RC and NP and rapidly decreases with the bio-linker length Δ (FIG. 21).

One should also comment on the quantum yield of the hybrid complex, defined as

$${Y}_{HS} = \frac{{R}_{HS}}{{R}_{abs}} , where {R}_{abs} = \frac{{I}_{0}}{ℏω} ( {σ}_{RC} + {σ}_{NP} )$$

is the total rate of photon absorption by the hybrid system. As mentioned above, due to the very short relaxation times (τ_{RC,relax}˜30 ps and τ_{P700→Chl}=1 ps), the RC itself has a very high quantum yield Y_{RC}≈1. Therefore, main losses in the hybrid system come from recombination of excitons in a NP and the yield becomes

$${Y}_{HS} \approx \frac{{γ}_{Foerster}}{( {γ}_{NP}^{0} + {γ}_{Foerster} )} ;$$

the above equation was obtained under the conditions σ_{NP}>>σ_{RC} and γ_{NP}⁰˜γ_{Foerster}. It can be seen that the resultant quantum yield becomes relatively low: Y_{HS}≈0.33 for the bio-linker with Δ=1 nm. However, the total rate of production of electrons strongly increased: R_{tot,HS}/R_{tot,HS}≈77. Basically, the reduced quantum yield is “a price” for the strongly enhanced absorption.

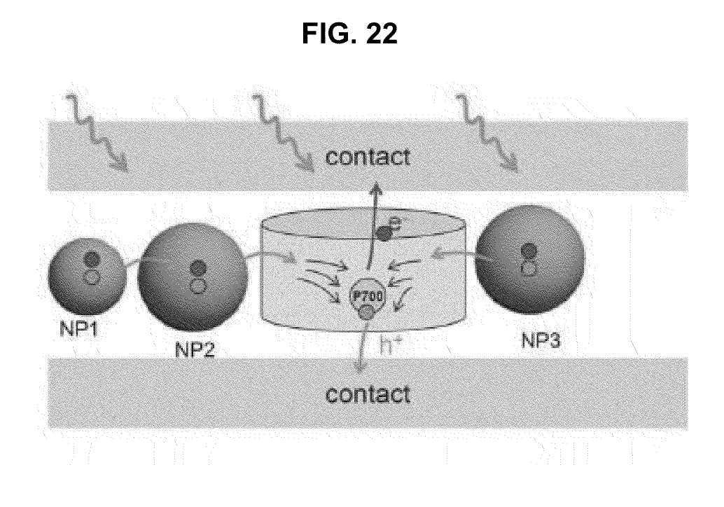

In the final part of this example, advantages and disadvantages of hybrid nanostructure which comprises a semiconductor NP and PS I molecule are discussed. The quantum yield strongly depends on the geometry of the system. For example, for the same NP with R_{NP}=4 nm, the calculated yield strongly increases if we decrease the distance to the RC: Y_{HS}≈0.54 for Δ=0. Another way to increase Y_{HS} is to decrease the NP radius (inset of FIG. 21). But, in this case, we also decrease the absorption by a NP and therefore R_{HS} decreases. However, few or several smaller NPs can be attached to one RC and, in this way, R_{HS} can be increased keeping a decent value of Y_{HS}. For example, Y_{HS}≈0.53 if we choose R_{NP}=3 nm, Δ=1 nm, and the geometry shown in FIG. 18B. The geometry convenient for photocurrent experiments is shown in FIG. 22, it resembles the system studied experimentally in R. Das, P. J. Kiley, M. Segal, J. Norville, A. A. Yu, L. Wang, A. S. Trammell, L. E. Reddick, R. Kumar, F. Stellacci, N. Lebedev, J. Schnur, B. D Bruce, S. Zhang, M. Baldo, Nano Lett. 2004, 4, 1079. In the structure shown in FIG. 22, a photo-generated electron and hole become first separated inside the RC and then forwarded to the conducting contacts. The resultant photocurrent is proportional to R_{HS}. The role of semiconductor NPs in this structure is to enhance the absorption cross section and to supply excitons to the RC. The role of RC is to rapidly separate the electron and hole. The structure in FIG. 22 includes three NPs and a RC. In particular, excitons generated in the NP1 are channeled to the RC via the NP2. The NPs 1 and 2 communicate via the FT mechanism. The NPs 2 and 3 are coupled with the RC again via the FT process. The rate of generation of excitons is:

$${R}_{HS} = \frac{{I}_{0}}{ℏω} ( \begin{matrix}{σ}_{RC} + 2 {σ}_{NP 2} \frac{{γ}_{Foerster , NP 2 \to RC}}{( {γ}_{NP}^{0} + {γ}_{Foerster , NP 2 \to RC} )} + \\ {σ}_{NP 1} \frac{{γ}_{Foerster , NP 1 \to NP 2}}{( {γ}_{NP}^{0} + {γ}_{Foerster , NP 2 \to NP 2} )} \frac{{γ}_{Foerster , NP 2 \to RC}}{( {γ}_{NP}^{0} + {γ}_{Foerster , NP 2 \to RC} )}\end{matrix} )$$

where γ_{Foerste,NP1→NP2} is the rate for the FT process NP1→NP2. For simplicity, it may be assumed that all NPs have the same γ_{NP}⁰. A similar cascade transfer was studied theoretically and realized in J. Lee, A. O. Govorov, N. A. Kotov, Nano Lett. 2005, 5, 2063. The rate of FT between NPs can be estimated by

$${γ}_{Foerster , NP 1 \to NP 2} \approx \frac{{V}_{NP 2}}{πℏ} {( \frac{{ed}_{exc}}{{ɛ}_{eff}} )}^{2} {\vert \frac{3 {ɛ}_{0}}{2 {ɛ}_{0} + {ɛ}_{NP} ( {ω}_{exc , NP 2} )} \vert}^{2} \frac{1}{{d}^{6}} Im {ɛ}_{NP} ( {ω}_{exc , NP 2} ) ,$$

where d is the center-to-center distance between NPs and ω_{exc,NP2} is the frequency of NP2. The following parameters can now be assumed: R_{NP1}=3 nm, R_{NP2}=4 nm, Δ_{NP1-NP2}=Δ_{NP2-RC}=1 nm, where Δ_{NP1-NP2(NP2-RC)} are the surface-to-surface spatial separations. The calculated FT rates are: γ_{Foerster,NP1→NP1}/γ_{NP}⁰=17 and γ_{Foerster,NP2→RC}/γ_{NP}⁰=0.29. It can be seen that the slowest step is the process is NP2→RC. The step NP1→NP2 is fast (1/γ_{Foerster,NP1→NP1}˜1.2 ns) and does not lead to significant losses of energy. For the structure in FIG. 22, the quantum yield Y_{HS}≈0.37. For the same structure and wavelength interval 360 nm <λ<550 nm, we obtain R_{tot,HS}/R_{tot,RS}≈115.

The hybrid nanostructure of the present embodiments has a strongly increased rate of generation of electrons because the absorption cross section of a semiconductor NP is much larger than that for a RC. Simultaneously, the interaction between a NP and RC via Förster transfer results in enhanced opto-electronic properties. A NP in the hybrid nanostructure of the present embodiments delivers excitons to a RC where they become separated within a very short time. Of note, the quantum yield of the hybrid nanostructure becomes reduced. This happens due to recombination of excitons in a NP. The present modeling shows that excitons of a NP recombine since the time of FT from a NP to a RC is relatively long. The reasons for the relatively long time for the process NP→RC are the following: (1) The absorption cross section of a RC is not very large; (2) the NP-RC center-to-center distance is relatively long since it is dictated by the size of the RC. In order to increase the quantum yield of a hybrid structure, the effective distance between the NP and RC is preferably decreased. This can be done, for example, using NPs with smaller sizes or shorter bio-linkers. Possible ways to increase the rate of production of electrons are illustrated in FIG. 22. To obtain an enhanced rate R_{HS}, one can use few or several NPs attached to a RC or one can assemble chains of NPs with cascade energy transfer. For light-harvesting applications, the hybrid structure of the present embodiment is particularly advantageous if one monolayer or a thin film is used. The amount of light energy absorbed by a thin film composed of hybrid complexes will be greatly enhanced.

The natural photosynthesis systems typically include both reaction centers and antenna chlorophylls. These components (RC and antenna chlorophylls) are built into a membrane that holds the system components. The antenna chlorophylls serve to absorb photons and to deliver them to the reaction center via the FT mechanism. In the system with reaction centers and antenna chlorophylls, the enhancement effect can also be achieved by attaching semiconductor NPs to the antennas. Experimentally, NPs can be attached to a membrane that contains photosystems and the enhancement effect can be observed as an increased rate of production of a chemical “fuel” (e.g. ATP molecules).

Natural photosystems absorb light mostly within certain wavelength intervals. A hybrid nanostructure composed of a photosystem and semiconductor nanoparticles in accordance with some embodiments of the present invention can efficiently harvest light energy in a much wider wavelength interval. Moreover, the rate of production of excited electrons by the hybrid nanostructure of the present embodiments is enhanced compared to the photosystem alone. The amount of enhancement also depends on the geometry of hybrid nanostructure. A judicial selection of the geometry can provide enhancement of up to one hundred times.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A nanostructure comprising at least one semiconductor nanoparticle bound to a photocatalytic unit of a photosynthetic organism, wherein said nanoparticle and a binding between said nanoparticle and said photocatalytic unit are selected such that transfer of electrons from said photocatalytic unit to said nanoparticle is prevented or suppressed relative to transfer of excitons from said nanoparticle to said photocatalytic unit.

2. The nanostructure of claim 1, wherein said at least one semiconductor nanoparticle binds to a polypeptide of a reaction center of said photocatalytic unit.

3. The nanostructure of claim 1, wherein said at least one semiconductor nanoparticle binds to an antenna chlorophyll of said photocatalytic unit.

4. The nanostructure of claim 1, wherein said photosynthetic organism is a green plant.

5. The nanostructure of claim 1, wherein said photosynthetic organism is a cyanobacteria.

6. The nanostructure of claim 1, wherein said photocatalytic unit is photosystem I (PS I).

7. The nanostructure of claim 5, wherein said photosynthetic organism is a Synechosystis sp. PCC 6803.

8-9. (canceled)

10. The nanostructure of claim 1, wherein said at least one semiconductor nanoparticle is binds to said photocatalytic unit via a bifunctional connecting molecule.

11-13. (canceled)

14. The nanostructure of claim 1, wherein a polypeptide of said photocatalytic unit comprises at least one substitution mutation.

15-18. (canceled)

19. The nanostructure of claim 14, wherein said polypeptide comprises an amino acid sequence is as set forth in SEQ ID NOs: 1, 2, 3, 4, 5 and 6.

20. The nanostructure of claim 1, wherein a diameter of said at least one semiconductor nanoparticle is about 2 nm to 20 nm.

21. The nanostructure of claim 1, wherein a diameter of said at least one semiconductor nanoparticle is about 8 nm.

22. (canceled)

23. The nanostructure of claim 1, wherein said semiconductor nanoparticle is selected from the group consisting of a CdTe nanoparticle, a CdSe nanoparticle, and a CdS nanoparticle.

24. A device comprising a nanostructure, attached to at least one electrode, wherein said nanostructure comprises at least one semiconductor nanoparticle bound to a photocatalytic unit of a photosynthetic organism, wherein said nanoparticle and a binding between said nanoparticle and said photocatalytic unit are selected such that transfer of electrons from said photocatalytic unit to said nanoparticle is prevented or suppressed relative to transfer of excitons from said nanoparticle to said photocatalytic unit.

25. The device of claim 24, wherein said electrode comprises a transition metal.

26. (canceled)

27. The device of claim 24, serving as a component selected from the group consisting of a photodiode, a phototransistor, a logic gate, a solar cell and an optocoupler.

28. A method of fabricating a device, the method comprising:
(a) covalently attaching photosystem I (PSI) of a photosynthetic organism to a solid support to generate a monolayer of photocatalytic units;
(b) depositing platinum ions on said monolayer under conditions that allow generation of a platinized monolayer of said photocatalytic units:
(c) depositing free, pre-plantinized PSIs of said photosynthetic organism on said monolayer of said photocatalytic units to generate a multilayered assembly of said photocatalytic units, wherein a polypeptide of said pre-platinized PSIs comprises at least one cysteine substitution mutation, thereby fabricating the device.

29. The method of claim 28, wherein the device serves as a component selected from the group consisting of a photodiode, a phototransistor, a logic gate, a solar cell and an optocoupler.

30. The method of claim 28, wherein said conditions comprise incubation in light in the presence of an electron donor.

31. (canceled)


SEQUENCE LISTING (10 sequences)

SEQ ID NO: 1 - protein, 731 aa, Synechocystis sp.
  misc_feature: D480C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPCSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPYGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 2 - protein, 731 aa, Synechocystis sp.
  misc_feature: S500C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINCGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPYGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 3 - protein, 731 aa, Synechocystis sp.
  misc_feature: S600C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWCGNVAQFNENSTYLMGWFRDYLWANSAQLINGYNPYGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 4 - protein, 731 aa, Synechocystis sp.
  misc_feature: Y635C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPCGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 5 - protein, 731 aa, Synechocystis sp.
  misc_feature: D236C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAACPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPYGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 6 - protein, 731 aa, Synechocystis sp.
  misc_feature: S247C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTCEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPYGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 7 - protein, 81 aa, Synechocystis sp.
  misc_feature: W32C mutant polypeptide
MSHSVKIYDTCIGCTQCVRACPLDVLEMVPCDGCKAAQIASSPRTEDCVGCKRCETACPTDFLSIRVYLGAETTRSMGLAY

SEQ ID NO: 8 - protein, 812 aa, Synechocystis sp.
  misc_feature: W32C/D236C/Y635C mutant polypeptide
MSHSVKIYDTCIGCTQCVRACPLDVLEMVPCDGCKAAQIASSPRTEDCVGCKRCETACPTDFLSIRVYLGAETTRSMGLAYMATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAACPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPCGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 9 - protein, 731 aa, Synechocystis sp.
  misc_feature: D236C/Y635C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAACPDTAGHIFGTSEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPCGVNNLSVWAWMFLFGHLVWATGFMFLISWRGYWQELIETIVWAHERTPLANLVRWKDKPVALSIVQARLVGLAHFTVGYVLTYAAFLIASTAGKFG

SEQ ID NO: 10 - protein, 731 aa, Synechocystis sp.
  misc_feature: S247C/Y635C mutant polypeptide
MATKFPKFSQDLAQDPTTRRIWYGIATAHDFETHDGMTEENLYQKIFASHFGHIAIIFLWTSGTLFHVAWQGNFEQWIKDPLNIRPIAHAIWDPHFGEGAVNAFTQAGASNPVNIAYSGVYHWFYTIGMTTNQELYSGAVFLLVLASLFLFAGWLHLQPKFRPSLAWFKNAESRLNHHLAGLFGVSSLAWAGHLVHVAIPEARGQHVGWDNFLSTPPHPAGLMPFFTGNWGVYAADPDTAGHIFGTCEGAGTAILTFLGGFHPQTESLWLTDIAHHHLAIAVIFIIAGHMYRTNWGIGHSIKEILNAHKGPLTGAGHTNLYDTINNSLHFQLGLALASLGVITSLVAQHMYSLPSYAFIAQDHTTQAALYTHHQYIAGFLMVGAFAHGAIFFVRDYDPVANKDNVLARMLEHKEALISHLSWVSLFLGFHTLGLYVHNDVVVAFGTPEKQILIEPVFAQWIQATSGKALYGFDVLLSNPDSIASTTGAAWLPGWLDAINSGTNSLFLTIGPGDFLVHHAIALGLHTTALILIKGALDARGSKLMPDKKDFGYSFPCDGPGRGGTCDISAWDAFYLAMFWMLNTLGWLTFYWHWKHLGVWSGNVAQFNENSTYLMGWFRDYLWANSAQLINGYNPCGVNNLSVWAWMFLFGHLVWATGFMFLISWRGYWQELIETIVWAHERTPLANLVRWKDKPVALSIVQARLVGLAHFTVGYVLTYAAFLIASTAGKFG